SEQUENCE LISTING (41 sequences)

SEQ ID NO: 1 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 2 - DNA, 3156 bp, Staphylococcus aureus
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 3 - RNA, 3156 nt, Staphylococcus aureus
aagcggaacuacauccugggccuggacaucggcaucaccagcgugggcuacggcaucaucgacuacgagacacgggacgugaucgaugccggcgugcggcuguucaaagaggccaacguggaaaacaacgagggcaggcggagcaagagaggcgccagaaggcugaagcggcggaggcggcauagaauccagagagugaagaagcugcuguucgacuacaaccugcugaccgaccacagcgagcugagcggcaucaaccccuacgaggccagagugaagggccugagccagaagcugagcgaggaagaguucucugccgcccugcugcaccuggccaagagaagaggcgugcacaacgugaacgagguggaagaggacaccggcaacgagcuguccaccaaagagcagaucagccggaacagcaaggcccuggaagagaaauacguggccgaacugcagcuggaacggcugaagaaagacggcgaagugcggggcagcaucaacagauucaagaccagcgacuacgugaaagaagccaaacagcugcugaaggugcagaaggccuaccaccagcuggaccagagcuucaucgacaccuacaucgaccugcuggaaacccggcggaccuacuaugagggaccuggcgagggcagccccuucggcuggaaggacaucaaagaaugguacgagaugcugaugggccacugcaccuacuuccccgaggaacugcggagcgugaaguacgccuacaacgccgaccuguacaacgcccugaacgaccugaacaaucucgugaucaccagggacgagaacgagaagcuggaauauuacgagaaguuccagaucaucgagaacguguucaagcagaagaagaagcccacccugaagcagaucgccaaagaaauccucgugaacgaagaggauauuaagggcuacagagugaccagcaccggcaagcccgaguucaccaaccugaagguguaccacgacaucaaggacauuaccgcccggaaagagauuauugagaacgccgagcugcuggaucagauugccaagauccugaccaucuaccagagcagcgaggacauccaggaagaacugaccaaucugaacuccgagcugacccaggaagagaucgagcagaucucuaaucugaagggcuauaccggcacccacaaccugagccugaaggccaucaaccugauccuggacgagcuguggcacaccaacgacaaccagaucgcuaucuucaaccggcugaagcuggugcccaagaagguggaccugucccagcagaaagagauccccaccacccugguggacgacuucauccugagccccgucgugaagagaagcuucauccagagcaucaaagugaucaacgccaucaucaagaaguacggccugcccaacgacaucauuaucgagcuggcccgcgagaagaacuccaaggacgcccagaaaaugaucaacgagaugcagaagcggaaccggcagaccaacgagcggaucgaggaaaucauccggaccaccggcaaagagaacgccaaguaccugaucgagaagaucaagcugcacgacaugcaggaaggcaagugccuguacagccuggaagccaucccucuggaagaucugcugaacaaccccuucaacuaugagguggaccacaucauccccagaagcguguccuucgacaacagcuucaacaacaaggugcucgugaagcaggaagaaaacagcaagaagggcaaccggaccccauuccaguaccugagcagcagcgacagcaagaucagcuacgaaaccuucaagaagcacauccugaaucuggccaagggcaagggcagaaucagcaagaccaagaaagaguaucugcuggaagaacgggacaucaacagguucuccgugcagaaagacuucaucaaccggaaccugguggauaccagauacgccaccagaggccugaugaaccugcugcggagcuacuucagagugaacaaccuggacgugaaagugaaguccaucaauggcggcuucaccagcuuucugcggcggaaguggaaguuuaagaaagagcggaacaagggguacaagcaccacgccgaggacgcccugaucauugccaacgccgauuucaucuucaaagaguggaagaaacuggacaaggccaaaaaagugauggaaaaccagauguucgaggaaaagcaggccgagagcaugcccgagaucgaaaccgagcaggaguacaaagagaucuucaucaccccccaccagaucaagcacauuaaggacuucaaggacuacaaguacagccaccggguggacaagaagccuaauagagagcugauuaacgacacccuguacuccacccggaaggacgacaagggcaacacccugaucgugaacaaucugaacggccuguacgacaaggacaaugacaagcugaaaaagcugaucaacaagagccccgaaaagcugcugauguaccaccacgacccccagaccuaccagaaacugaagcugauuauggaacaguacggcgacgagaagaauccccuguacaaguacuacgaggaaaccgggaacuaccugaccaaguacuccaaaaaggacaacggccccgugaucaagaagauuaaguauuacggcaacaaacugaacgcccaucuggacaucaccgacgacuaccccaacagcagaaacaaggucgugaagcugucccugaagcccuacagauucgacguguaccuggacaauggcguguacaaguucgugaccgugaagaaucuggaugugaucaaaaaagaaaacuacuacgaagugaauagcaagugcuaugaggaagcuaagaagcugaagaagaucagcaaccaggccgaguuuaucgccuccuucuacaacaacgaucugaucaagaucaacggcgagcuguauagagugaucggcgugaacaacgaccugcugaaccggaucgaagugaacaugaucgacaucaccuaccgcgaguaccuggaaaacaugaacgacaagaggccccccaggaucauuaagacaaucgccuccaagacccagagcauuaagaaguacagcacagacauucugggcaaccuguaugaagugaaaucuaagaagcacccucagaucaucaaaaagggc

SEQ ID NO: 4 - DNA, 3156 bp, Artificial Sequence
  Synthetic
aagcggaactacatcctgggcctggacatcggcatcaccagcgtgggctacggcatcatcgactacgagactcgtgatgttattgacgcaggcgttcgtttgtttaaagaagctaatgttgagaataatgagggaagaagaagtaagcgtggggctcgcaggcttaagcgaagaagaaggcatcggatacagcgtgtgaagaagttgctgtttgattataatttgttgactgatcattctgagttatcaggcattaatccttatgaggctcgtgttaagggtttaagtcagaagttaagtgaagaagaattttctgctgctttgttgcatttggctaaaagaagaggagttcataatgttaatgaagttgaagaggatactggtaatgagttaagtactaaggagcagataagtcgtaattctaaggctttggaagaaaagtatgttgctgagttgcagttggagcgtttgaagaaggatggtgaagtaagaggaagtattaatcgttttaagacaagtgattatgtgaaagaagcgaagcagttgttgaaagttcagaaggcttatcatcagttggatcaaagttttattgatacttatattgatttgttggagactcgtagaacttattatgagggtcctggtgaggggtccccgtttggttggaaggatattaaggagtggtatgagatgttgatgggtcattgtacttattttcctgaagaattgcggtccgtgaagtatgcttataatgctgatttgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 5 - RNA, 3156 nt, Artificial Sequence
  Synthetic
aagcggaacuacauccugggccuggacaucggcaucaccagcgugggcuacggcaucaucgacuacgagacucgugauguuauugacgcaggcguucguuuguuuaaagaagcuaauguugagaauaaugagggaagaagaaguaagcguggggcucgcaggcuuaagcgaagaagaaggcaucggauacagcgugugaagaaguugcuguuugauuauaauuuguugacugaucauucugaguuaucaggcauuaauccuuaugaggcucguguuaaggguuuaagucagaaguuaagugaagaagaauuuucugcugcuuuguugcauuuggcuaaaagaagaggaguucauaauguuaaugaaguugaagaggauacugguaaugaguuaaguacuaaggagcagauaagucguaauucuaaggcuuuggaagaaaaguauguugcugaguugcaguuggagcguuugaagaaggauggugaaguaagaggaaguauuaaucguuuuaagacaagugauuaugugaaagaagcgaagcaguuguugaaaguucagaaggcuuaucaucaguuggaucaaaguuuuauugauacuuauauugauuuguuggagacucguagaacuuauuaugaggguccuggugagggguccccguuugguuggaaggauauuaaggagugguaugagauguugaugggucauuguacuuauuuuccugaagaauugcgguccgugaaguaugcuuauaaugcugauuuguacaacgcccugaacgaccugaacaaucucgugaucaccagggacgagaacgagaagcuggaauauuacgagaaguuccagaucaucgagaacguguucaagcagaagaagaagcccacccugaagcagaucgccaaagaaauccucgugaacgaagaggauauuaagggcuacagagugaccagcaccggcaagcccgaguucaccaaccugaagguguaccacgacaucaaggacauuaccgcccggaaagagauuauugagaacgccgagcugcuggaucagauugccaagauccugaccaucuaccagagcagcgaggacauccaggaagaacugaccaaucugaacuccgagcugacccaggaagagaucgagcagaucucuaaucugaagggcuauaccggcacccacaaccugagccugaaggccaucaaccugauccuggacgagcuguggcacaccaacgacaaccagaucgcuaucuucaaccggcugaagcuggugcccaagaagguggaccugucccagcagaaagagauccccaccacccugguggacgacuucauccugagccccgucgugaagagaagcuucauccagagcaucaaagugaucaacgccaucaucaagaaguacggccugcccaacgacaucauuaucgagcuggcccgcgagaagaacuccaaggacgcccagaaaaugaucaacgagaugcagaagcggaaccggcagaccaacgagcggaucgaggaaaucauccggaccaccggcaaagagaacgccaaguaccugaucgagaagaucaagcugcacgacaugcaggaaggcaagugccuguacagccuggaagccaucccucuggaagaucugcugaacaaccccuucaacuaugagguggaccacaucauccccagaagcguguccuucgacaacagcuucaacaacaaggugcucgugaagcaggaagaaaacagcaagaagggcaaccggaccccauuccaguaccugagcagcagcgacagcaagaucagcuacgaaaccuucaagaagcacauccugaaucuggccaagggcaagggcagaaucagcaagaccaagaaagaguaucugcuggaagaacgggacaucaacagguucuccgugcagaaagacuucaucaaccggaaccugguggauaccagauacgccaccagaggccugaugaaccugcugcggagcuacuucagagugaacaaccuggacgugaaagugaaguccaucaauggcggcuucaccagcuuucugcggcggaaguggaaguuuaagaaagagcggaacaagggguacaagcaccacgccgaggacgcccugaucauugccaacgccgauuucaucuucaaagaguggaagaaacuggacaaggccaaaaaagugauggaaaaccagauguucgaggaaaagcaggccgagagcaugcccgagaucgaaaccgagcaggaguacaaagagaucuucaucaccccccaccagaucaagcacauuaaggacuucaaggacuacaaguacagccaccggguggacaagaagccuaauagagagcugauuaacgacacccuguacuccacccggaaggacgacaagggcaacacccugaucgugaacaaucugaacggccuguacgacaaggacaaugacaagcugaaaaagcugaucaacaagagccccgaaaagcugcugauguaccaccacgacccccagaccuaccagaaacugaagcugauuauggaacaguacggcgacgagaagaauccccuguacaaguacuacgaggaaaccgggaacuaccugaccaaguacuccaaaaaggacaacggccccgugaucaagaagauuaaguauuacggcaacaaacugaacgcccaucuggacaucaccgacgacuaccccaacagcagaaacaaggucgugaagcugucccugaagcccuacagauucgacguguaccuggacaauggcguguacaaguucgugaccgugaagaaucuggaugugaucaaaaaagaaaacuacuacgaagugaauagcaagugcuaugaggaagcuaagaagcugaagaagaucagcaaccaggccgaguuuaucgccuccuucuacaacaacgaucugaucaagaucaacggcgagcuguauagagugaucggcgugaacaacgaccugcugaaccggaucgaagugaacaugaucgacaucaccuaccgcgaguaccuggaaaacaugaacgacaagaggccccccaggaucauuaagacaaucgccuccaagacccagagcauuaagaaguacagcacagacauucugggcaaccuguaugaagugaaaucuaagaagcacccucagaucaucaaaaagggc

SEQ ID NO: 6 - DNA, 3156 bp, Artificial Sequence
  Synthetic
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgctaagattttgactatttatcagtcaagtgaggatattcaggaagaattgactaatttgaattctgagttgactcaggaagaaattgagcagataagtaatttgaagggatacactggtactcataatttaagtttgaaggctattaatttgattttggatgagttgtggcatactaatgataatcagattgctatttttaatcgtttgaagttggttcctaagaaagttgatttaagtcagcagaaggagattcctactactttggttgatgactttattttaagtcctgttgttaagcgaagttttattcaaagtattaaagttattaatgctattattaagaagtatgggctcccgaatgatattattattgagttggctcgtgagaagaattctaaagatgctcagaagatgattaatgagatgcagaagaggaacagacagacaaatgaaagaattgaagaaattattcggacaactggtaaggagaatgctaagtatttgattgagaagattaagttgcatgatatgcaggagggtaagtgtttgtattctttggaggctattcctttggaggatttgttgaataatccttttaattatgaagttgatcatattattcctcggtccgtaagttttgataattcttttaataataaagttttggttaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 7 - RNA, 3156 nt, Artificial Sequence
  Synthetic
aagcggaacuacauccugggccuggacaucggcaucaccagcgugggcuacggcaucaucgacuacgagacacgggacgugaucgaugccggcgugcggcuguucaaagaggccaacguggaaaacaacgagggcaggcggagcaagagaggcgccagaaggcugaagcggcggaggcggcauagaauccagagagugaagaagcugcuguucgacuacaaccugcugaccgaccacagcgagcugagcggcaucaaccccuacgaggccagagugaagggccugagccagaagcugagcgaggaagaguucucugccgcccugcugcaccuggccaagagaagaggcgugcacaacgugaacgagguggaagaggacaccggcaacgagcuguccaccaaagagcagaucagccggaacagcaaggcccuggaagagaaauacguggccgaacugcagcuggaacggcugaagaaagacggcgaagugcggggcagcaucaacagauucaagaccagcgacuacgugaaagaagccaaacagcugcugaaggugcagaaggccuaccaccagcuggaccagagcuucaucgacaccuacaucgaccugcuggaaacccggcggaccuacuaugagggaccuggcgagggcagccccuucggcuggaaggacaucaaagaaugguacgagaugcugaugggccacugcaccuacuuccccgaggaacugcggagcgugaaguacgccuacaacgccgaccuguacaacgcccugaacgaccugaacaaucucgugaucaccagggacgagaacgagaagcuggaauauuacgagaaguuccagaucaucgagaacguguucaagcagaagaagaagcccacccugaagcagaucgccaaagaaauccucgugaacgaagaggauauuaagggcuacagagugaccagcaccggcaagcccgaguucaccaaccugaagguguaccacgacaucaaggacauuaccgcccggaaagagauuauugagaacgccgagcugcuggaucagauugcuaagauuuugacuauuuaucagucaagugaggauauucaggaagaauugacuaauuugaauucugaguugacucaggaagaaauugagcagauaaguaauuugaagggauacacugguacucauaauuuaaguuugaaggcuauuaauuugauuuuggaugaguuguggcauacuaaugauaaucagauugcuauuuuuaaucguuugaaguugguuccuaagaaaguugauuuaagucagcagaaggagauuccuacuacuuugguugaugacuuuauuuuaaguccuguuguuaagcgaaguuuuauucaaaguauuaaaguuauuaaugcuauuauuaagaaguaugggcucccgaaugauauuauuauugaguuggcucgugagaagaauucuaaagaugcucagaagaugauuaaugagaugcagaagaggaacagacagacaaaugaaagaauugaagaaauuauucggacaacugguaaggagaaugcuaaguauuugauugagaagauuaaguugcaugauaugcaggaggguaaguguuuguauucuuuggaggcuauuccuuuggaggauuuguugaauaauccuuuuaauuaugaaguugaucauauuauuccucgguccguaaguuuugauaauucuuuuaauaauaaaguuuugguuaagcaggaagaaaacagcaagaagggcaaccggaccccauuccaguaccugagcagcagcgacagcaagaucagcuacgaaaccuucaagaagcacauccugaaucuggccaagggcaagggcagaaucagcaagaccaagaaagaguaucugcuggaagaacgggacaucaacagguucuccgugcagaaagacuucaucaaccggaaccugguggauaccagauacgccaccagaggccugaugaaccugcugcggagcuacuucagagugaacaaccuggacgugaaagugaaguccaucaauggcggcuucaccagcuuucugcggcggaaguggaaguuuaagaaagagcggaacaagggguacaagcaccacgccgaggacgcccugaucauugccaacgccgauuucaucuucaaagaguggaagaaacuggacaaggccaaaaaagugauggaaaaccagauguucgaggaaaagcaggccgagagcaugcccgagaucgaaaccgagcaggaguacaaagagaucuucaucaccccccaccagaucaagcacauuaaggacuucaaggacuacaaguacagccaccggguggacaagaagccuaauagagagcugauuaacgacacccuguacuccacccggaaggacgacaagggcaacacccugaucgugaacaaucugaacggccuguacgacaaggacaaugacaagcugaaaaagcugaucaacaagagccccgaaaagcugcugauguaccaccacgacccccagaccuaccagaaacugaagcugauuauggaacaguacggcgacgagaagaauccccuguacaaguacuacgaggaaaccgggaacuaccugaccaaguacuccaaaaaggacaacggccccgugaucaagaagauuaaguauuacggcaacaaacugaacgcccaucuggacaucaccgacgacuaccccaacagcagaaacaaggucgugaagcugucccugaagcccuacagauucgacguguaccuggacaauggcguguacaaguucgugaccgugaagaaucuggaugugaucaaaaaagaaaacuacuacgaagugaauagcaagugcuaugaggaagcuaagaagcugaagaagaucagcaaccaggccgaguuuaucgccuccuucuacaacaacgaucugaucaagaucaacggcgagcuguauagagugaucggcgugaacaacgaccugcugaaccggaucgaagugaacaugaucgacaucaccuaccgcgaguaccuggaaaacaugaacgacaagaggccccccaggaucauuaagacaaucgccuccaagacccagagcauuaagaaguacagcacagacauucugggcaaccuguaugaagugaaaucuaagaagcacccucagaucaucaaaaagggc

SEQ ID NO: 8 - DNA, 3156 bp, Artificial Sequence
  synthetic
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtataaggagatttttataacacctcatcagattaagcatattaaggattttaaggattataagtattctcatcgtgtggacaagaagcctaatcgtgagttgattaatgatactttgtattcgactcgtaaggatgacaaaggtaacaccttgattgttaataatttgaatggtttgtatgataaggacaatgataagttgaagaagttgattaataagtctcctgagaagttgttgatgtatcatcatgatccgcagacttatcagaagttgaagttgattatggagcagtatggtgatgagaagaatcctttgtataagtattatgaagaaactggtaattatttgactaagtattcgaagaaggacaatgggcccgtgattaagaagattaagtattatggtaataagttgaatgctcatttggatattactgatgactatcctaattctcgtaataaagttgttaagttaagtttgaagccttatcgttttgatgtttatttggacaatggtgtttataagtttgttactgtgaagaatttggatgttattaagaaggagaattattatgaagttaattctaagtgttatgaagaagcgaagaagttgaagaagataagtaatcaggctgagtttattgcaagtttttataataatgatttgattaagattaatggtgagttgtatcgtgttattggtgttaataatgatttgttgaatcgtattgaagttaatatgattgatattacttatcgtgagtatttggagaatatgaatgataagcggcccccgcgtattattaagactattgcaagtaagactcaaagtattaagaagtattctactgatattttgggtaatttgtatgaagttaagtcgaagaagcatcctcagattattaagaagggt

SEQ ID NO: 9 - RNA, 3156 nt, Artificial Sequence
  Synthetic
aagcggaacuacauccugggccuggacaucggcaucaccagcgugggcuacggcaucaucgacuacgagacacgggacgugaucgaugccggcgugcggcuguucaaagaggccaacguggaaaacaacgagggcaggcggagcaagagaggcgccagaaggcugaagcggcggaggcggcauagaauccagagagugaagaagcugcuguucgacuacaaccugcugaccgaccacagcgagcugagcggcaucaaccccuacgaggccagagugaagggccugagccagaagcugagcgaggaagaguucucugccgcccugcugcaccuggccaagagaagaggcgugcacaacgugaacgagguggaagaggacaccggcaacgagcuguccaccaaagagcagaucagccggaacagcaaggcccuggaagagaaauacguggccgaacugcagcuggaacggcugaagaaagacggcgaagugcggggcagcaucaacagauucaagaccagcgacuacgugaaagaagccaaacagcugcugaaggugcagaaggccuaccaccagcuggaccagagcuucaucgacaccuacaucgaccugcuggaaacccggcggaccuacuaugagggaccuggcgagggcagccccuucggcuggaaggacaucaaagaaugguacgagaugcugaugggccacugcaccuacuuccccgaggaacugcggagcgugaaguacgccuacaacgccgaccuguacaacgcccugaacgaccugaacaaucucgugaucaccagggacgagaacgagaagcuggaauauuacgagaaguuccagaucaucgagaacguguucaagcagaagaagaagcccacccugaagcagaucgccaaagaaauccucgugaacgaagaggauauuaagggcuacagagugaccagcaccggcaagcccgaguucaccaaccugaagguguaccacgacaucaaggacauuaccgcccggaaagagauuauugagaacgccgagcugcuggaucagauugccaagauccugaccaucuaccagagcagcgaggacauccaggaagaacugaccaaucugaacuccgagcugacccaggaagagaucgagcagaucucuaaucugaagggcuauaccggcacccacaaccugagccugaaggccaucaaccugauccuggacgagcuguggcacaccaacgacaaccagaucgcuaucuucaaccggcugaagcuggugcccaagaagguggaccugucccagcagaaagagauccccaccacccugguggacgacuucauccugagccccgucgugaagagaagcuucauccagagcaucaaagugaucaacgccaucaucaagaaguacggccugcccaacgacaucauuaucgagcuggcccgcgagaagaacuccaaggacgcccagaaaaugaucaacgagaugcagaagcggaaccggcagaccaacgagcggaucgaggaaaucauccggaccaccggcaaagagaacgccaaguaccugaucgagaagaucaagcugcacgacaugcaggaaggcaagugccuguacagccuggaagccaucccucuggaagaucugcugaacaaccccuucaacuaugagguggaccacaucauccccagaagcguguccuucgacaacagcuucaacaacaaggugcucgugaagcaggaagaaaacagcaagaagggcaaccggaccccauuccaguaccugagcagcagcgacagcaagaucagcuacgaaaccuucaagaagcacauccugaaucuggccaagggcaagggcagaaucagcaagaccaagaaagaguaucugcuggaagaacgggacaucaacagguucuccgugcagaaagacuucaucaaccggaaccugguggauaccagauacgccaccagaggccugaugaaccugcugcggagcuacuucagagugaacaaccuggacgugaaagugaaguccaucaauggcggcuucaccagcuuucugcggcggaaguggaaguuuaagaaagagcggaacaagggguacaagcaccacgccgaggacgcccugaucauugccaacgccgauuucaucuucaaagaguggaagaaacuggacaaggccaaaaaagugauggaaaaccagauguucgaggaaaagcaggccgagagcaugcccgagaucgaaaccgagcaggaguauaaggagauuuuuauaacaccucaucagauuaagcauauuaaggauuuuaaggauuauaaguauucucaucguguggacaagaagccuaaucgugaguugauuaaugauacuuuguauucgacucguaaggaugacaaagguaacaccuugauuguuaauaauuugaaugguuuguaugauaaggacaaugauaaguugaagaaguugauuaauaagucuccugagaaguuguugauguaucaucaugauccgcagacuuaucagaaguugaaguugauuauggagcaguauggugaugagaagaauccuuuguauaaguauuaugaagaaacugguaauuauuugacuaaguauucgaagaaggacaaugggcccgugauuaagaagauuaaguauuaugguaauaaguugaaugcucauuuggauauuacugaugacuauccuaauucucguaauaaaguuguuaaguuaaguuugaagccuuaucguuuugauguuuauuuggacaaugguguuuauaaguuuguuacugugaagaauuuggauguuauuaagaaggagaauuauuaugaaguuaauucuaaguguuaugaagaagcgaagaaguugaagaagauaaguaaucaggcugaguuuauugcaaguuuuuauaauaaugauuugauuaagauuaauggugaguuguaucguguuauugguguuaauaaugauuuguugaaucguauugaaguuaauaugauugauauuacuuaucgugaguauuuggagaauaugaaugauaagcggcccccgcguauuauuaagacuauugcaaguaagacucaaaguauuaagaaguauucuacugauauuuuggguaauuuguaugaaguuaagucgaagaagcauccucagauuauuaagaagggu

SEQ ID NO: 10 - DNA, 693 bp, Artificial Sequence
  Synthetic
actcgtgatgttattgacgcaggcgttcgtttgtttaaagaagctaatgttgagaataatgagggaagaagaagtaagcgtggggctcgcaggcttaagcgaagaagaaggcatcggatacagcgtgtgaagaagttgctgtttgattataatttgttgactgatcattctgagttatcaggcattaatccttatgaggctcgtgttaagggtttaagtcagaagttaagtgaagaagaattttctgctgctttgttgcatttggctaaaagaagaggagttcataatgttaatgaagttgaagaggatactggtaatgagttaagtactaaggagcagataagtcgtaattctaaggctttggaagaaaagtatgttgctgagttgcagttggagcgtttgaagaaggatggtgaagtaagaggaagtattaatcgttttaagacaagtgattatgtgaaagaagcgaagcagttgttgaaagttcagaaggcttatcatcagttggatcaaagttttattgatacttatattgatttgttggagactcgtagaacttattatgagggtcctggtgaggggtccccgtttggttggaaggatattaaggagtggtatgagatgttgatgggtcattgtacttattttcctgaagaattgcggtccgtgaagtatgcttataatgctgatttg

SEQ ID NO: 11 - RNA, 693 nt, Artificial Sequence
  Synthetic
acucgugauguuauugacgcaggcguucguuuguuuaaagaagcuaauguugagaauaaugagggaagaagaaguaagcguggggcucgcaggcuuaagcgaagaagaaggcaucggauacagcgugugaagaaguugcuguuugauuauaauuuguugacugaucauucugaguuaucaggcauuaauccuuaugaggcucguguuaaggguuuaagucagaaguuaagugaagaagaauuuucugcugcuuuguugcauuuggcuaaaagaagaggaguucauaauguuaaugaaguugaagaggauacugguaaugaguuaaguacuaaggagcagauaagucguaauucuaaggcuuuggaagaaaaguauguugcugaguugcaguuggagcguuugaagaaggauggugaaguaagaggaaguauuaaucguuuuaagacaagugauuaugugaaagaagcgaagcaguuguugaaaguucagaaggcuuaucaucaguuggaucaaaguuuuauugauacuuauauugauuuguuggagacucguagaacuuauuaugaggguccuggugagggguccccguuugguuggaaggauauuaaggagugguaugagauguugaugggucauuguacuuauuuuccugaagaauugcgguccgugaaguaugcuuauaaugcugauuug

SEQ ID NO: 12 - DNA, 672 bp, Artificial Sequence
  Synthetic
gctaagattttgactatttatcagtcaagtgaggatattcaggaagaattgactaatttgaattctgagttgactcaggaagaaattgagcagataagtaatttgaagggatacactggtactcataatttaagtttgaaggctattaatttgattttggatgagttgtggcatactaatgataatcagattgctatttttaatcgtttgaagttggttcctaagaaagttgatttaagtcagcagaaggagattcctactactttggttgatgactttattttaagtcctgttgttaagcgaagttttattcaaagtattaaagttattaatgctattattaagaagtatgggctcccgaatgatattattattgagttggctcgtgagaagaattctaaagatgctcagaagatgattaatgagatgcagaagaggaacagacagacaaatgaaagaattgaagaaattattcggacaactggtaaggagaatgctaagtatttgattgagaagattaagttgcatgatatgcaggagggtaagtgtttgtattctttggaggctattcctttggaggatttgttgaataatccttttaattatgaagttgatcatattattcctcggtccgtaagttttgataattcttttaataataaagttttggtt

SEQ ID NO: 13 - RNA, 672 nt, Artificial Sequence
  Synthetic
gcuaagauuuugacuauuuaucagucaagugaggauauucaggaagaauugacuaauuugaauucugaguugacucaggaagaaauugagcagauaaguaauuugaagggauacacugguacucauaauuuaaguuugaaggcuauuaauuugauuuuggaugaguuguggcauacuaaugauaaucagauugcuauuuuuaaucguuugaaguugguuccuaagaaaguugauuuaagucagcagaaggagauuccuacuacuuugguugaugacuuuauuuuaaguccuguuguuaagcgaaguuuuauucaaaguauuaaaguuauuaaugcuauuauuaagaaguaugggcucccgaaugauauuauuauugaguuggcucgugagaagaauucuaaagaugcucagaagaugauuaaugagaugcagaagaggaacagacagacaaaugaaagaauugaagaaauuauucggacaacugguaaggagaaugcuaaguauuugauugagaagauuaaguugcaugauaugcaggaggguaaguguuuguauucuuuggaggcuauuccuuuggaggauuuguugaauaauccuuuuaauuaugaaguugaucauauuauuccucgguccguaaguuuugauaauucuuuuaauaauaaaguuuugguu

SEQ ID NO: 14 - DNA, 912 bp, Artificial Sequence
  Synthetic
tataaggagatttttataacacctcatcagattaagcatattaaggattttaaggattataagtattctcatcgtgtggacaagaagcctaatcgtgagttgattaatgatactttgtattcgactcgtaaggatgacaaaggtaacaccttgattgttaataatttgaatggtttgtatgataaggacaatgataagttgaagaagttgattaataagtctcctgagaagttgttgatgtatcatcatgatccgcagacttatcagaagttgaagttgattatggagcagtatggtgatgagaagaatcctttgtataagtattatgaagaaactggtaattatttgactaagtattcgaagaaggacaatgggcccgtgattaagaagattaagtattatggtaataagttgaatgctcatttggatattactgatgactatcctaattctcgtaataaagttgttaagttaagtttgaagccttatcgttttgatgtttatttggacaatggtgtttataagtttgttactgtgaagaatttggatgttattaagaaggagaattattatgaagttaattctaagtgttatgaagaagcgaagaagttgaagaagataagtaatcaggctgagtttattgcaagtttttataataatgatttgattaagattaatggtgagttgtatcgtgttattggtgttaataatgatttgttgaatcgtattgaagttaatatgattgatattacttatcgtgagtatttggagaatatgaatgataagcggcccccgcgtattattaagactattgcaagtaagactcaaagtattaagaagtattctactgatattttgggtaatttgtatgaagttaagtcgaagaagcatcctcagattattaagaagggt

SEQ ID NO: 15 - RNA, 912 nt, Artificial Sequence
  Synthetic
uauaaggagauuuuuauaacaccucaucagauuaagcauauuaaggauuuuaaggauuauaaguauucucaucguguggacaagaagccuaaucgugaguugauuaaugauacuuuguauucgacucguaaggaugacaaagguaacaccuugauuguuaauaauuugaaugguuuguaugauaaggacaaugauaaguugaagaaguugauuaauaagucuccugagaaguuguugauguaucaucaugauccgcagacuuaucagaaguugaaguugauuauggagcaguauggugaugagaagaauccuuuguauaaguauuaugaagaaacugguaauuauuugacuaaguauucgaagaaggacaaugggcccgugauuaagaagauuaaguauuaugguaauaaguugaaugcucauuuggauauuacugaugacuauccuaauucucguaauaaaguuguuaaguuaaguuugaagccuuaucguuuugauguuuauuuggacaaugguguuuauaaguuuguuacugugaagaauuuggauguuauuaagaaggagaauuauuaugaaguuaauucuaaguguuaugaagaagcgaagaaguugaagaagauaaguaaucaggcugaguuuauugcaaguuuuuauaauaaugauuugauuaagauuaauggugaguuguaucguguuauugguguuaauaaugauuuguugaaucguauugaaguuaauaugauugauauuacuuaucgugaguauuuggagaauaugaaugauaagcggcccccgcguauuauuaagacuauugcaaguaagacucaaaguauuaagaaguauucuacugauauuuuggguaauuuguaugaaguuaagucgaagaagcauccucagauuauuaagaagggu

SEQ ID NO: 16 - DNA, 69 bp, Artificial Sequence
  Synthetic
tttcaggcgctaaaacataccagatgaaagtctggagaggtgaagaatacgaccacctagcgcctgaaa

SEQ ID NO: 17 - RNA, 69 nt, Artificial Sequence
  Synthetic
uuucaggcgcuaaaacauaccagaugaaagucuggagaggugaagaauacgaccaccuagcgccugaaa

SEQ ID NO: 18 - DNA, 69 bp, Artificial Sequence
  Synthetic
tttcaggcgccaaaacataccagatgaaagtctggagaggtgaagaatacgaccacctggcgcctgaaa

SEQ ID NO: 19 - RNA, 69 nt, Artificial Sequence
  Synthetic
uuucaggcgccaaaacauaccagaugaaagucuggagaggugaagaauacgaccaccuggcgccugaaa

SEQ ID NO: 20 - DNA, 71 bp, Artificial Sequence
  Synthetic
tttcaggcgcgcaaaacataccagatgaaagtctggagaggtgaagaatacgaccacctgcgcgcctgaaa

SEQ ID NO: 21 - RNA, 71 nt, Artificial Sequence
  Synthetic
uuucaggcgcgcaaaacauaccagaugaaagucuggagaggugaagaauacgaccaccugcgcgccugaaa

SEQ ID NO: 22 - DNA, 96 bp, Artificial Sequence
  Synthetic
caaccaaacaaccaaacaaccaaacaaccaaacaaccaaacaaccaaacaaccaaacaaccaaacaaccaaacaaccaaacaaccaaacaacacag

SEQ ID NO: 23 - RNA, 96 nt, Artificial Sequence
  Synthetic
caaccaaacaaccaaacaaccaaacaaccaaacaaccaaacaaccaaacaaccaaacaaccaaacaaccaaacaaccaaacaaccaaacaacacag

SEQ ID NO: 24 - DNA, 101 bp, Artificial Sequence
  Synthetic
gtgagtctatgggacccttgatgttttctgcatgggtagccgctgagatggagcctgagcacacgcggccgctgttaacgcagtgtttctctttttttcag

SEQ ID NO: 25 - RNA, 101 nt, Artificial Sequence
  Synthetic
gugagucuaugggacccuugauguuuucugcauggguagccgcugagauggagccugagcacacgcggccgcuguuaacgcaguguuucucuuuuuuucag

SEQ ID NO: 26 - DNA, 91 bp, Artificial Sequence
  Synthetic
gttggtgctagctggccaaggctggattattctgagtccaagctaggcccttttgctaatcatgttcatacctcttatcttcctcccacag

SEQ ID NO: 27 - RNA, 91 nt, Artificial Sequence
  Synthetic
guuggugcuagcuggccaaggcuggauuauucugaguccaagcuaggcccuuuugcuaaucauguucauaccucuuaucuuccucccacag

SEQ ID NO: 28 - DNA, 351 bp, Artificial Sequence
  Synthetic
gtgagtctatgggacccttgatgttttttgcatgggtagccgctgagatggagcctgagcacacgcggccgctgttaacgcagtgtttctctttttttcaggcgctaaaacataccagatgaaagtctggagaggtgaagaatacgaccacctagcgcctgaaacaaccaaacaaccaaacaaccaaacaaccaaacaaccaaacaaccaaacaaccaaacaaccaaacaaccaaacaaccaaacaaccaaacaacacaggttggtgctagctggccaaggctggattattctgagtccaagctaggcccttttgctaatcatgttcatacctcttatcttcctcccacag

SEQ ID NO: 29 - RNA, 351 nt, Artificial Sequence
  Synthetic
gugagucuaugggacccuugauguuuuuugcauggguagccgcugagauggagccugagcacacgcggccgcuguuaacgcaguguuucucuuuuuuucaggcgcuaaaacauaccagaugaaagucuggagaggugaagaauacgaccaccuagcgccugaaacaaccaaacaaccaaacaaccaaacaaccaaacaaccaaacaaccaaacaaccaaacaaccaaacaaccaaacaaccaaacaaccaaacaacacagguuggugcuagcuggccaaggcuggauuauucugaguccaagcuaggcccuuuugcuaaucauguucauaccucuuaucuuccucccacag

SEQ ID NO: 30 - DNA, 3507 bp, Artificial Sequence
  Synthetic
aagcggaactacatcctgggcctggacatcggcatcaccagcgtgggctacggcatcatcgactacgagactcgtgatgttattgacgcaggcgttcgtttgtttaaagaagctaatgttgagaataatgagggaagaagaagtaagcgtggggctcgcaggcttagtgagtctatgggacccttgatgttttttgcatgggtagccgctgagatggagcctgagcacacgcggccgctgttaacgcagtgtttctctttttttcaggcgctaaaacataccagatgaaagtctggagaggtgaagaatacgaccacctagcgcctgaaacaaccaaacaaccaaacaaccaaacaaccaaacaaccaaacaaccaaacaaccaaacaaccaaacaaccaaacaaccaaacaaccaaacaacacaggttggtgctagctggccaaggctggattattctgagtccaagctaggcccttttgctaatcatgttcatacctcttatcttcctcccacagagcgaagaagaaggcatcggatacagcgtgtgaagaagttgctgtttgattataatttgttgactgatcattctgagttatcaggcattaatccttatgaggctcgtgttaagggtttaagtcagaagttaagtgaagaagaattttctgctgctttgttgcatttggctaaaagaagaggagttcataatgttaatgaagttgaagaggatactggtaatgagttaagtactaaggagcagataagtcgtaattctaaggctttggaagaaaagtatgttgctgagttgcagttggagcgtttgaagaaggatggtgaagtaagaggaagtattaatcgttttaagacaagtgattatgtgaaagaagcgaagcagttgttgaaagttcagaaggcttatcatcagttggatcaaagttttattgatacttatattgatttgttggagactcgtagaacttattatgagggtcctggtgaggggtccccgtttggttggaaggatattaaggagtggtatgagatgttgatgggtcattgtacttattttcctgaagaattgcggtccgtgaagtatgcttataatgctgatttgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 31 - RNA, 3507 nt, Artificial Sequence
  Synthetic
aagcggaacuacauccugggccuggacaucggcaucaccagcgugggcuacggcaucaucgacuacgagacucgugauguuauugacgcaggcguucguuuguuuaaagaagcuaauguugagaauaaugagggaagaagaaguaagcguggggcucgcaggcuuagugagucuaugggacccuugauguuuuuugcauggguagccgcugagauggagccugagcacacgcggccgcuguuaacgcaguguuucucuuuuuuucaggcgcuaaaacauaccagaugaaagucuggagaggugaagaauacgaccaccuagcgccugaaacaaccaaacaaccaaacaaccaaacaaccaaacaaccaaacaaccaaacaaccaaacaaccaaacaaccaaacaaccaaacaaccaaacaacacagguuggugcuagcuggccaaggcuggauuauucugaguccaagcuaggcccuuuugcuaaucauguucauaccucuuaucuuccucccacagagcgaagaagaaggcaucggauacagcgugugaagaaguugcuguuugauuauaauuuguugacugaucauucugaguuaucaggcauuaauccuuaugaggcucguguuaaggguuuaagucagaaguuaagugaagaagaauuuucugcugcuuuguugcauuuggcuaaaagaagaggaguucauaauguuaaugaaguugaagaggauacugguaaugaguuaaguacuaaggagcagauaagucguaauucuaaggcuuuggaagaaaaguauguugcugaguugcaguuggagcguuugaagaaggauggugaaguaagaggaaguauuaaucguuuuaagacaagugauuaugugaaagaagcgaagcaguuguugaaaguucagaaggcuuaucaucaguuggaucaaaguuuuauugauacuuauauugauuuguuggagacucguagaacuuauuaugaggguccuggugagggguccccguuugguuggaaggauauuaaggagugguaugagauguugaugggucauuguacuuauuuuccugaagaauugcgguccgugaaguaugcuuauaaugcugauuuguacaacgcccugaacgaccugaacaaucucgugaucaccagggacgagaacgagaagcuggaauauuacgagaaguuccagaucaucgagaacguguucaagcagaagaagaagcccacccugaagcagaucgccaaagaaauccucgugaacgaagaggauauuaagggcuacagagugaccagcaccggcaagcccgaguucaccaaccugaagguguaccacgacaucaaggacauuaccgcccggaaagagauuauugagaacgccgagcugcuggaucagauugccaagauccugaccaucuaccagagcagcgaggacauccaggaagaacugaccaaucugaacuccgagcugacccaggaagagaucgagcagaucucuaaucugaagggcuauaccggcacccacaaccugagccugaaggccaucaaccugauccuggacgagcuguggcacaccaacgacaaccagaucgcuaucuucaaccggcugaagcuggugcccaagaagguggaccugucccagcagaaagagauccccaccacccugguggacgacuucauccugagccccgucgugaagagaagcuucauccagagcaucaaagugaucaacgccaucaucaagaaguacggccugcccaacgacaucauuaucgagcuggcccgcgagaagaacuccaaggacgcccagaaaaugaucaacgagaugcagaagcggaaccggcagaccaacgagcggaucgaggaaaucauccggaccaccggcaaagagaacgccaaguaccugaucgagaagaucaagcugcacgacaugcaggaaggcaagugccuguacagccuggaagccaucccucuggaagaucugcugaacaaccccuucaacuaugagguggaccacaucauccccagaagcguguccuucgacaacagcuucaacaacaaggugcucgugaagcaggaagaaaacagcaagaagggcaaccggaccccauuccaguaccugagcagcagcgacagcaagaucagcuacgaaaccuucaagaagcacauccugaaucuggccaagggcaagggcagaaucagcaagaccaagaaagaguaucugcuggaagaacgggacaucaacagguucuccgugcagaaagacuucaucaaccggaaccugguggauaccagauacgccaccagaggccugaugaaccugcugcggagcuacuucagagugaacaaccuggacgugaaagugaaguccaucaauggcggcuucaccagcuuucugcggcggaaguggaaguuuaagaaagagcggaacaagggguacaagcaccacgccgaggacgcccugaucauugccaacgccgauuucaucuucaaagaguggaagaaacuggacaaggccaaaaaagugauggaaaaccagauguucgaggaaaagcaggccgagagcaugcccgagaucgaaaccgagcaggaguacaaagagaucuucaucaccccccaccagaucaagcacauuaaggacuucaaggacuacaaguacagccaccggguggacaagaagccuaauagagagcugauuaacgacacccuguacuccacccggaaggacgacaagggcaacacccugaucgugaacaaucugaacggccuguacgacaaggacaaugacaagcugaaaaagcugaucaacaagagccccgaaaagcugcugauguaccaccacgacccccagaccuaccagaaacugaagcugauuauggaacaguacggcgacgagaagaauccccuguacaaguacuacgaggaaaccgggaacuaccugaccaaguacuccaaaaaggacaacggccccgugaucaagaagauuaaguauuacggcaacaaacugaacgcccaucuggacaucaccgacgacuaccccaacagcagaaacaaggucgugaagcugucccugaagcccuacagauucgacguguaccuggacaauggcguguacaaguucgugaccgugaagaaucuggaugugaucaaaaaagaaaacuacuacgaagugaauagcaagugcuaugaggaagcuaagaagcugaagaagaucagcaaccaggccgaguuuaucgccuccuucuacaacaacgaucugaucaagaucaacggcgagcuguauagagugaucggcgugaacaacgaccugcugaaccggaucgaagugaacaugaucgacaucaccuaccgcgaguaccuggaaaacaugaacgacaagaggccccccaggaucauuaagacaaucgccuccaagacccagagcauuaagaaguacagcacagacauucugggcaaccuguaugaagugaaaucuaagaagcacccucagaucaucaaaaagggc

SEQ ID NO: 32 - DNA, 3507 bp, Artificial Sequence
  Synthetic
aagcggaactacatcctgggcctggacatcggcatcaccagcgtgggctacggcatcatcgactacgagactcgtgatgttattgacgcaggcgttcgtttgtttaaagaagctaatgttgagaataatgagggaagaagaagtaagcgtggggctcgcaggcttaagcgaagaagaaggcatcggatacagcgtgtgaagaagttgctgtttgattataatttgttgactgatcattctgagttatcaggcattaatccttatgaggctcgtgttaagggtttaagtcagaagttaagtgaagaagaattttctgctgctttgttgcatttggctaaaagaagaggagttcataatgttaatgaagttgaagaggatactggtaatgagttaagtactaaggagcagataagtcgtaattctaaggctttggaagaaaagtatgttgctgagttgcagttggagcgtttgaagaaggatggtgaagtaagaggaagtattaatcgttttaagacaagtgattatgtgaaagaagcgaagcagttgttgaaagttcagaaggcttatgtgagtctatgggacccttgatgttttctgcatgggtagccgctgagatggagcctgagcacacgcggccgctgttaacgcagtgtttctctttttttcaggcgctaaaacataccagatgaaagtctggagaggtgaagaatacgaccacctagcgcctgaaacaaccaaacaaccaaacaaccaaacaaccaaacaaccaaacaaccaaacaaccaaacaaccaaacaaccaaacaaccaaacaaccaaacaacacaggttggtgctagctggccaaggctggattattctgagtccaagctaggcccttttgctaatcatgttcatacctcttatcttcctcccacagcatcagttggatcaaagttttattgatacttatattgatttgttggagactcgtagaacttattatgagggtcctggtgaggggtccccgtttggttggaaggatattaaggagtggtatgagatgttgatgggtcattgtacttattttcctgaagaattgcggtccgtgaagtatgcttataatgctgatttgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 33 - RNA, 3507 nt, Artificial Sequence
  Synthetic
aagcggaacuacauccugggccuggacaucggcaucaccagcgugggcuacggcaucaucgacuacgagacucgugauguuauugacgcaggcguucguuuguuuaaagaagcuaauguugagaauaaugagggaagaagaaguaagcguggggcucgcaggcuuaagcgaagaagaaggcaucggauacagcgugugaagaaguugcuguuugauuauaauuuguugacugaucauucugaguuaucaggcauuaauccuuaugaggcucguguuaaggguuuaagucagaaguuaagugaagaagaauuuucugcugcuuuguugcauuuggcuaaaagaagaggaguucauaauguuaaugaaguugaagaggauacugguaaugaguuaaguacuaaggagcagauaagucguaauucuaaggcuuuggaagaaaaguauguugcugaguugcaguuggagcguuugaagaaggauggugaaguaagaggaaguauuaaucguuuuaagacaagugauuaugugaaagaagcgaagcaguuguugaaaguucagaaggcuuaugugagucuaugggacccuugauguuuucugcauggguagccgcugagauggagccugagcacacgcggccgcuguuaacgcaguguuucucuuuuuuucaggcgcuaaaacauaccagaugaaagucuggagaggugaagaauacgaccaccuagcgccugaaacaaccaaacaaccaaacaaccaaacaaccaaacaaccaaacaaccaaacaaccaaacaaccaaacaaccaaacaaccaaacaaccaaacaacacagguuggugcuagcuggccaaggcuggauuauucugaguccaagcuaggcccuuuugcuaaucauguucauaccucuuaucuuccucccacagcaucaguuggaucaaaguuuuauugauacuuauauugauuuguuggagacucguagaacuuauuaugaggguccuggugagggguccccguuugguuggaaggauauuaaggagugguaugagauguugaugggucauuguacuuauuuuccugaagaauugcgguccgugaaguaugcuuauaaugcugauuuguacaacgcccugaacgaccugaacaaucucgugaucaccagggacgagaacgagaagcuggaauauuacgagaaguuccagaucaucgagaacguguucaagcagaagaagaagcccacccugaagcagaucgccaaagaaauccucgugaacgaagaggauauuaagggcuacagagugaccagcaccggcaagcccgaguucaccaaccugaagguguaccacgacaucaaggacauuaccgcccggaaagagauuauugagaacgccgagcugcuggaucagauugccaagauccugaccaucuaccagagcagcgaggacauccaggaagaacugaccaaucugaacuccgagcugacccaggaagagaucgagcagaucucuaaucugaagggcuauaccggcacccacaaccugagccugaaggccaucaaccugauccuggacgagcuguggcacaccaacgacaaccagaucgcuaucuucaaccggcugaagcuggugcccaagaagguggaccugucccagcagaaagagauccccaccacccugguggacgacuucauccugagccccgucgugaagagaagcuucauccagagcaucaaagugaucaacgccaucaucaagaaguacggccugcccaacgacaucauuaucgagcuggcccgcgagaagaacuccaaggacgcccagaaaaugaucaacgagaugcagaagcggaaccggcagaccaacgagcggaucgaggaaaucauccggaccaccggcaaagagaacgccaaguaccugaucgagaagaucaagcugcacgacaugcaggaaggcaagugccuguacagccuggaagccaucccucuggaagaucugcugaacaaccccuucaacuaugagguggaccacaucauccccagaagcguguccuucgacaacagcuucaacaacaaggugcucgugaagcaggaagaaaacagcaagaagggcaaccggaccccauuccaguaccugagcagcagcgacagcaagaucagcuacgaaaccuucaagaagcacauccugaaucuggccaagggcaagggcagaaucagcaagaccaagaaagaguaucugcuggaagaacgggacaucaacagguucuccgugcagaaagacuucaucaaccggaaccugguggauaccagauacgccaccagaggccugaugaaccugcugcggagcuacuucagagugaacaaccuggacgugaaagugaaguccaucaauggcggcuucaccagcuuucugcggcggaaguggaaguuuaagaaagagcggaacaagggguacaagcaccacgccgaggacgcccugaucauugccaacgccgauuucaucuucaaagaguggaagaaacuggacaaggccaaaaaagugauggaaaaccagauguucgaggaaaagcaggccgagagcaugcccgagaucgaaaccgagcaggaguacaaagagaucuucaucaccccccaccagaucaagcacauuaaggacuucaaggacuacaaguacagccaccggguggacaagaagccuaauagagagcugauuaacgacacccuguacuccacccggaaggacgacaagggcaacacccugaucgugaacaaucugaacggccuguacgacaaggacaaugacaagcugaaaaagcugaucaacaagagccccgaaaagcugcugauguaccaccacgacccccagaccuaccagaaacugaagcugauuauggaacaguacggcgacgagaagaauccccuguacaaguacuacgaggaaaccgggaacuaccugaccaaguacuccaaaaaggacaacggccccgugaucaagaagauuaaguauuacggcaacaaacugaacgcccaucuggacaucaccgacgacuaccccaacagcagaaacaaggucgugaagcugucccugaagcccuacagauucgacguguaccuggacaauggcguguacaaguucgugaccgugaagaaucuggaugugaucaaaaaagaaaacuacuacgaagugaauagcaagugcuaugaggaagcuaagaagcugaagaagaucagcaaccaggccgaguuuaucgccuccuucuacaacaacgaucugaucaagaucaacggcgagcuguauagagugaucggcgugaacaacgaccugcugaaccggaucgaagugaacaugaucgacaucaccuaccgcgaguaccuggaaaacaugaacgacaagaggccccccaggaucauuaagacaaucgccuccaagacccagagcauuaagaaguacagcacagacauucugggcaaccuguaugaagugaaaucuaagaagcacccucagaucaucaaaaagggc

SEQ ID NO: 34 - DNA, 3858 bp, Artificial Sequence
  synthetic
aagcggaactacatcctgggcctggacatcggcatcaccagcgtgggctacggcatcatcgactacgagactcgtgatgttattgacgcaggcgttcgtttgtttaaagaagctaatgttgagaataatgagggaagaagaagtaagcgtggggctcgcaggcttagtgagtctatgggacccttgatgttttttgcatgggtagccgctgagatggagcctgagcacacgcggccgctgttaacgcagtgtttctctttttttcaggcgctaaaacataccagatgaaagtctggagaggtgaagaatacgaccacctagcgcctgaaacaaccaaacaaccaaacaaccaaacaaccaaacaaccaaacaaccaaacaaccaaacaaccaaacaaccaaacaaccaaacaaccaaacaacacaggttggtgctagctggccaaggctggattattctgagtccaagctaggcccttttgctaatcatgttcatacctcttatcttcctcccacagagcgaagaagaaggcatcggatacagcgtgtgaagaagttgctgtttgattataatttgttgactgatcattctgagttatcaggcattaatccttatgaggctcgtgttaagggtttaagtcagaagttaagtgaagaagaattttctgctgctttgttgcatttggctaaaagaagaggagttcataatgttaatgaagttgaagaggatactggtaatgagttaagtactaaggagcagataagtcgtaattctaaggctttggaagaaaagtatgttgctgagttgcagttggagcgtttgaagaaggatggtgaagtaagaggaagtattaatcgttttaagacaagtgattatgtgaaagaagcgaagcagttgttgaaagttcagaaggcttatgtgagtctatgggacccttgatgttttctgcatgggtagccgctgagatggagcctgagcacacgcggccgctgttaacgcagtgtttctctttttttcaggcgctaaaacataccagatgaaagtctggagaggtgaagaatacgaccacctagcgcctgaaacaaccaaacaaccaaacaaccaaacaaccaaacaaccaaacaaccaaacaaccaaacaaccaaacaaccaaacaaccaaacaaccaaacaacacaggttggtgctagctggccaaggctggattattctgagtccaagctaggcccttttgctaatcatgttcatacctcttatcttcctcccacagcatcagttggatcaaagttttattgatacttatattgatttgttggagactcgtagaacttattatgagggtcctggtgaggggtccccgtttggttggaaggatattaaggagtggtatgagatgttgatgggtcattgtacttattttcctgaagaattgcggtccgtgaagtatgcttataatgctgatttgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 35 - RNA, 3858 nt, Artificial Sequence
  Synthetic
aagcggaacuacauccugggccuggacaucggcaucaccagcgugggcuacggcaucaucgacuacgagacucgugauguuauugacgcaggcguucguuuguuuaaagaagcuaauguugagaauaaugagggaagaagaaguaagcguggggcucgcaggcuuagugagucuaugggacccuugauguuuuuugcauggguagccgcugagauggagccugagcacacgcggccgcuguuaacgcaguguuucucuuuuuuucaggcgcuaaaacauaccagaugaaagucuggagaggugaagaauacgaccaccuagcgccugaaacaaccaaacaaccaaacaaccaaacaaccaaacaaccaaacaaccaaacaaccaaacaaccaaacaaccaaacaaccaaacaaccaaacaacacagguuggugcuagcuggccaaggcuggauuauucugaguccaagcuaggcccuuuugcuaaucauguucauaccucuuaucuuccucccacagagcgaagaagaaggcaucggauacagcgugugaagaaguugcuguuugauuauaauuuguugacugaucauucugaguuaucaggcauuaauccuuaugaggcucguguuaaggguuuaagucagaaguuaagugaagaagaauuuucugcugcuuuguugcauuuggcuaaaagaagaggaguucauaauguuaaugaaguugaagaggauacugguaaugaguuaaguacuaaggagcagauaagucguaauucuaaggcuuuggaagaaaaguauguugcugaguugcaguuggagcguuugaagaaggauggugaaguaagaggaaguauuaaucguuuuaagacaagugauuaugugaaagaagcgaagcaguuguugaaaguucagaaggcuuaugugagucuaugggacccuugauguuuucugcauggguagccgcugagauggagccugagcacacgcggccgcuguuaacgcaguguuucucuuuuuuucaggcgcuaaaacauaccagaugaaagucuggagaggugaagaauacgaccaccuagcgccugaaacaaccaaacaaccaaacaaccaaacaaccaaacaaccaaacaaccaaacaaccaaacaaccaaacaaccaaacaaccaaacaaccaaacaacacagguuggugcuagcuggccaaggcuggauuauucugaguccaagcuaggcccuuuugcuaaucauguucauaccucuuaucuuccucccacagcaucaguuggaucaaaguuuuauugauacuuauauugauuuguuggagacucguagaacuuauuaugaggguccuggugagggguccccguuugguuggaaggauauuaaggagugguaugagauguugaugggucauuguacuuauuuuccugaagaauugcgguccgugaaguaugcuuauaaugcugauuuguacaacgcccugaacgaccugaacaaucucgugaucaccagggacgagaacgagaagcuggaauauuacgagaaguuccagaucaucgagaacguguucaagcagaagaagaagcccacccugaagcagaucgccaaagaaauccucgugaacgaagaggauauuaagggcuacagagugaccagcaccggcaagcccgaguucaccaaccugaagguguaccacgacaucaaggacauuaccgcccggaaagagauuauugagaacgccgagcugcuggaucagauugccaagauccugaccaucuaccagagcagcgaggacauccaggaagaacugaccaaucugaacuccgagcugacccaggaagagaucgagcagaucucuaaucugaagggcuauaccggcacccacaaccugagccugaaggccaucaaccugauccuggacgagcuguggcacaccaacgacaaccagaucgcuaucuucaaccggcugaagcuggugcccaagaagguggaccugucccagcagaaagagauccccaccacccugguggacgacuucauccugagccccgucgugaagagaagcuucauccagagcaucaaagugaucaacgccaucaucaagaaguacggccugcccaacgacaucauuaucgagcuggcccgcgagaagaacuccaaggacgcccagaaaaugaucaacgagaugcagaagcggaaccggcagaccaacgagcggaucgaggaaaucauccggaccaccggcaaagagaacgccaaguaccugaucgagaagaucaagcugcacgacaugcaggaaggcaagugccuguacagccuggaagccaucccucuggaagaucugcugaacaaccccuucaacuaugagguggaccacaucauccccagaagcguguccuucgacaacagcuucaacaacaaggugcucgugaagcaggaagaaaacagcaagaagggcaaccggaccccauuccaguaccugagcagcagcgacagcaagaucagcuacgaaaccuucaagaagcacauccugaaucuggccaagggcaagggcagaaucagcaagaccaagaaagaguaucugcuggaagaacgggacaucaacagguucuccgugcagaaagacuucaucaaccggaaccugguggauaccagauacgccaccagaggccugaugaaccugcugcggagcuacuucagagugaacaaccuggacgugaaagugaaguccaucaauggcggcuucaccagcuuucugcggcggaaguggaaguuuaagaaagagcggaacaagggguacaagcaccacgccgaggacgcccugaucauugccaacgccgauuucaucuucaaagaguggaagaaacuggacaaggccaaaaaagugauggaaaaccagauguucgaggaaaagcaggccgagagcaugcccgagaucgaaaccgagcaggaguacaaagagaucuucaucaccccccaccagaucaagcacauuaaggacuucaaggacuacaaguacagccaccggguggacaagaagccuaauagagagcugauuaacgacacccuguacuccacccggaaggacgacaagggcaacacccugaucgugaacaaucugaacggccuguacgacaaggacaaugacaagcugaaaaagcugaucaacaagagccccgaaaagcugcugauguaccaccacgacccccagaccuaccagaaacugaagcugauuauggaacaguacggcgacgagaagaauccccuguacaaguacuacgaggaaaccgggaacuaccugaccaaguacuccaaaaaggacaacggccccgugaucaagaagauuaaguauuacggcaacaaacugaacgcccaucuggacaucaccgacgacuaccccaacagcagaaacaaggucgugaagcugucccugaagcccuacagauucgacguguaccuggacaauggcguguacaaguucgugaccgugaagaaucuggaugugaucaaaaaagaaaacuacuacgaagugaauagcaagugcuaugaggaagcuaagaagcugaagaagaucagcaaccaggccgaguuuaucgccuccuucuacaacaacgaucugaucaagaucaacggcgagcuguauagagugaucggcgugaacaacgaccugcugaaccggaucgaagugaacaugaucgacaucaccuaccgcgaguaccuggaaaacaugaacgacaagaggccccccaggaucauuaagacaaucgccuccaagacccagagcauuaagaaguacagcacagacauucugggcaaccuguaugaagugaaaucuaagaagcacccucagaucaucaaaaagggc

SEQ ID NO: 36 - DNA, 21 bp, Artificial Sequence
  Synthetic
ccaaagaagaagcggaaggtc

SEQ ID NO: 37 - RNA, 21 nt, Artificial Sequence
  Synthetic
ccaaagaagaagcggaagguc

SEQ ID NO: 38 - DNA, 54 bp, Artificial Sequence
  Synthetic
aaaaggccggcggccacgaaaaaggccggccaggcaaaaaagaaaaagggatcc

SEQ ID NO: 39 - RNA, 54 nt, Artificial Sequence
  Synthetic
aaaaggccggcggccacgaaaaaggccggccaggcaaaaaagaaaaagggaucc

SEQ ID NO: 40 - DNA, 27 bp, Artificial Sequence
  Synthetic
tacccatacgatgttccagattacgct

SEQ ID NO: 41 - RNA, 27 nt, Artificial Sequence
  Synthetic
uacccauacgauguuccagauuacgcu

(19) United States
(12) Patent Application Publication
(10) Pub. No.: US 2022/0127642 A1
(43) Pub. Date: Apr. 28, 2022

(54) CONTROLLABLE GENOME EDITING SYSTEM

(71) Applicant: APPLIED STEMCELL, INC., Milpitas, CA (US)

(72) Inventors: Ruby Yanru TSAI, San Jose, CA (US); Alfonso Pedro FARRUGGIO, Stanford, CA (US); Armon KHOSRAVIANI, Los Gatos, CA (US); Naisargi Shaileshkumar PATEL, San Jose, CA (US); Ling-Jie KONG, Union City, CA (US)

(21) Appl. No.: 17/427,099

(22) PCT Filed: Jan. 30, 2020
(86) PCT No.: PCT/US20/15974

§ 371 Date: Jul. 30, 2021

Related U.S. Application Data
(60) Provisional application No. 62/798,478, filed on Jan. 30, 2019.

(51) Int. Cl. C12N 15/86; C12N 9/22; C12N 15/66

(52) U.S. Cl. CPC C12N 15/86; C12N 15/66; C12N 9/22

(57) ABSTRACT

Provided herein are compositions and methods for genome editing and modification. In one embodiment, the composition comprises a regulatory gene expression construct that comprises a nucleic acid encoding an RNA comprising a sequence encoding a genome editing enzyme and a regulatory cassette operably linked to the sequence. In one embodiment, the regulatory cassette comprises a conditional exon and an aptamer domain which is capable of binding to an effector molecule to trigger a structural change of the RNA, thereby regulating splicing of the conditional exon and expression of the genome editing enzyme.

### CROSS-REFERENCE TO RELATED APPLICATIONS

This application claims priority to U.S. Provisional Application No. 62/798,478, filed Jan. 30, 2019, the disclosure of which is incorporated herein by reference.

### SEQUENCE LISTING

The sequence listing that is contained in the file named “044903-8025WO01-SL-20200130_ST25”, which is 85 KB (as measured in Microsoft Windows) and was created on Jan. 30, 2020, is filed herewith by electronic submission and is incorporated by reference herein.

### BACKGROUND

### I. Field of the Invention

The present invention generally relates to compositions and methods for genome editing and modification.

### II. Description of Related Art

Genome editing technology has revolutionized the biomedical field by allowing the site-specific insertion, deletion, modification or replacement of DNA in the genome of a living organism. Currently, the common methods of genome editing use engineered site-specific nucleases that create double-strand breaks at desired location in the genome. The induced double-strand breaks are repaired through homologous recombination or nonhomologous end-joining, resulting in targeted genome alteration.

While the current genome editing technology provides a powerful tool for site-specific genome alteration, off-target editing resulted from nonspecific and unintended cleavage by the engineered site-specific nuclease still remains a big concern. For example, multiple studies using early versions of CRISPR-Cas9 system found that more than 50% of RNA-guided endonuclease induced mutation were not occurring on-target (Fu et al. (2013) Nature Biotechnology, 31:822-6; Lin et al (2014) Nucleic Acid Research, 42:7473-85). It is concerned that the off-target effects may disrupt vital coding regions, leading to genotoxic effects such as cancer if the genome editing technology is used in therapeutics.

One of the major factors that contribute to off-target editing is the prolonged presence of the site-specific nuclease in the cell. The longer such site-specific nuclease remains active in a cell after gene-editing, the greater chances for off-target editing. Accordingly, several approaches have been attempted to control the activity of the site-specific nuclease in the cell by introducing on and off switch. For example, the Bondy-Denomy group used a naturally occurring bacteriophage protein that inhibits Cas9 immunity (Borges A L et al., Cell (2018) 174: 917-25). The David Liu group used inducible Cas9 based on small molecule activated intein (Davis K M et al., Nat Chem Biol. (2015) 11: 316-18). The Feng Zhang group at Broad Institute created a Cas9 protein that can be split into rapamycin sensitive dimerization domains (Zetsche B et al., Nat Biotechnol. (2015) 33: 139-42). However, such approaches introduce into the cell additional foreign protein that may be harmful. Therefore, there is a continuing need to develop new controllable system for genome editing.

### SUMMARY OF THE INVENTION

In one aspect, the present disclosure provides a composition for genome editing and modification. In one embodiment, the composition comprises a regulatory gene expression construct that comprises a nucleic acid encoding an RNA comprising a sequence encoding a genome editing enzyme and a regulatory cassette operably linked to the sequence.

In one embodiment, the regulatory cassette comprises a conditional exon and an aptamer domain which is capable of binding to an effector molecule to trigger a structural change of the RNA, thereby regulating splicing of the conditional exon and expression of the genome editing enzyme. In certain embodiments, the conditional exon is skipped during the splicing in the presence of the effector molecule.

In certain embodiments, the genome editing enzyme is expressed in a cell when the construct is delivered to the cell in the presence of the effector molecule. In one embodiment, the genome editing enzyme has a sequence of at least 90% (e.g. 90%, 95%, 98%, 99%) identity to SEQ ID NO: 1.

In one embodiment, the sequence encoding the genome editing enzyme is optimized to comprise an exonic splicing enhancer (ESE). In certain embodiments, the sequence encoding the genome editing enzyme contains an ESE optimized region having a sequence of at least 90% (e.g. 90%, 95%, 98%, 99%) identity to SEQ ID NO: 10, 12 or 14 in the DNA form or SEQ ID NO: 11, 13 or 15 in the RNA form.

In one embodiment, the sequence encoding the genome editing enzyme is at least 90% (e.g. 90%, 95%, 98%, 99%) identity to SEQ ID NO: 4, 6 or 8 in the DNA form or SEQ ID NO: 5, 7 or 9 in the RNA form.

In one embodiment, the aptamer domain has a sequence of at least 90% (e.g. 90%, 95%, 98%, 99%) identity to SEQ ID NO: 16, 18 or 20 in the DNA form or SEQ ID NO: 17, 19 or 21 in the RNA form.

In one embodiment, the conditional exon has a sequence of at least 90% (e.g. 90%, 95%, 98%, 99%) identity to SEQ ID NO: 22 in the DNA form or SEQ ID NO: 23 in the RNA form.

In one embodiment, the conditional exon is flanked by an upstream intron and a downstream intron. In one embodiment, the upstream intron has a sequence of at least 90% (e.g. 90%, 95%, 98%, 99%) identity to SEQ ID NO: 24 in the DNA form or SEQ ID NO: 25 in the RNA form. In one embodiment, the downstream intron has a sequence of at least 90% (e.g. 90%, 95%, 98%, 99%) identity to SEQ ID NO: 26 in the DNA form or SEQ ID NO: 27 in the RNA form.

In one embodiment, the regulatory cassette comprises a sequence of at least 90% (e.g. 90%, 95%, 98%, 99%) identity to SEQ ID NO: 28 in the DNA form or SEQ ID NO: 29 in the RNA form. In certain embodiments, the regulatory cassette is inserted between nucleotide position 97 and 98 of SEQ ID NO: 10 in the DNA form or between nucleotide position 498 and 499 of SEQ ID NO: 10 in the DNA form. In certain embodiment, the regulatable gene expression construct contains two regulatory cassettes, which are inserted at between nucleotide position 97 and 98 of SEQ ID NO: 10 and between nucleotide position 498 and 499 of SEQ ID NO: 10, respectively.

In one embodiment, the construct comprises a sequence of at least 90% (e.g. 90%, 95%, 98%, 99%) identity to SEQ ID NO: 30, 32 or 34.

In one embodiment, the regulatory cassette includes a region capable of being recognized by a miRNA when the aptamer domain does not bind to the effector molecule, resulting the RNA being degraded. When the aptamer domain binds to the effector molecule, the structural change of the RNA prevents the region from being recognized by the miRNA, resulting in the expression of the genome editing enzyme. In one example, the effector molecule is tetracycline.

In certain embodiments, the genome editing enzyme is expressed in the cell in the absence of the effector molecule. In certain embodiment, the regulatory cassette inhibits the expression of the genome editing enzyme in the presence of the effector molecule.

In one embodiment, the regulatory cassette forms an anti-terminator stem when the aptamer domain does not bind to the effector molecule, thereby expressing the genome editing enzyme. When the aptamer domain binds to the effector molecule, the regulatory cassette forms a terminator stem, thereby inhibiting the expression of the genome editing enzyme.

In one embodiment, the regulatory cassette comprises a ribosome binding sequence that is recognized by ribosome when the aptamer domain does not bind to the effector molecule, thereby expressing the gene editing enzyme. When the aptamer domain binds to the effector molecule, the ribosome binding sequence is sequestered from being recognized by ribosome, thereby inhibiting the expression of the genome editing enzyme.

In certain embodiments, the effector molecule is a metabolite, e.g., adenosylcobalamin, aquocobalamin, cAMP, cGMP, c-di-AMP, c-di-GMP, fluoride, falvin mononucleotide, glutamine, glycine, lysine, nickel, cobalt, pre-queuosine, purine, S-adenosyl methionine, tetrahydrofolate, thiamin pyrophosphate, guanine, adenine, 2′-deoxyguanosine, 7-aminomethyl-7-deazaguanine, ZMP and ZTP.

In certain embodiments, the genome editing enzyme is a site-specific nuclease or a site-specific recombinase. In some embodiments, the site-specific nuclease is selected from a group consisting of Cas9, Cas12, ZFN, TALEN and meganuclease. In some embodiments, the site-specific recombinase is selected from a group consisting of Cre, FLP, lamda integrase, phiC31 integrase, Bxb1 integrase, gamma-delta resolvase, Tn3 resolvase and Gin invertase.

In certain embodiments, the construct is contained in a vector. In one example, the vector is an AAV vector.

In one embodiment, the gene editing enzyme is Cas9, and the nucleic acid construct further comprises a second polynucleotide sequence encoding a gRNA.

In another aspect, the present disclosure provides a method of genome editing in a cell. In one embodiment, the method comprises delivering the construct disclosed herein into the cell. In one embodiment, the method further comprises delivering the effector molecule to the cell.

In yet another aspect, the present disclosure provides a modified cell made by delivering the construct described herein into the cell.

In another aspect, the present disclosure provides a method of treating a subject having a disease. In one embodiment, the method comprises delivering the construct disclosed herein into at least one cell of the subject. In one embodiment, the method further comprises administering the effector molecule to the subject.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrates an exemplary embodiment of the nucleic acid construct of the present invention that the structural change of the RNA transcript regulates the splicing of the RNA transcript.

FIG. 2 illustrates an exemplary embodiment of the nucleic construct of the present invention that the nucleic acid construct encodes a Cas9 protein and is included in an AAV vector.

FIG. 3 illustrates an exemplary embodiment of the nucleic acid construct that the structural change of the RNA transcript regulates the stability of the RNA transcript.

FIG. 4 illustrates an exemplary embodiment of the nucleic acid construct of the present invention that the structural change of the RNA transcript regulates the translation of the RNA transcript.

FIG. 5 illustrates an exemplary embodiment of the nucleic acid construct of the present invention that the structural change of the RNA transcript regulates the translation of the RNA transcript.

FIG. 6 illustrates the addition of intron into SaCas9 gene.

FIG. 7 illustrates a schematic of the SaCas9 construct in which a SaCas9 gene is under the control of CMV promoter. The SaCas9 gene may be optimized with ESE enrichment and ESS depletion and contain one or more introns, an aptamer and a conditional exon.

FIG. 8 illustrates the results of the EGxxFP assay of the SaCas9 gene with addition of intron.

FIG. 9 illustrates the results of the EGxxFP assay of the SaCas9 gene containing an aptamer domain and a conditional exon.

FIG. 10 illustrates the results of the EGxxFP assay of the SaCas9 gene with dual aptamer domains in the absence of tetracycline.

FIG. 11 illustrates the results of the EGxxFP assay of the SaCas9 gene with dual aptamer domains in the presence of tetracycline.

### DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### I. DEFINITION

As used herein, the singular forms “a”, “an” and “the” include plural references unless the context clearly dictates otherwise.

It is noted that in this disclosure, terms such as “comprises”, “comprised”, “comprising”, “contains”, “containing” and the like are inclusive or open-ended and do not exclude additional, un-recited elements or method steps. Terms such as “consisting essentially of” and “consists essentially of” allow for the inclusion of additional ingredients or steps that do not materially affect the basic and novel characteristics of the claimed invention. The terms “consists of” and “consisting of” are close ended.

The term “aptamer” refers to a nucleotide sequence that can bind specifically to a target molecule. Aptamers are usually created by selection from a large random sequence pool, but also exist naturally, such as in riboswitches.

A “cell”, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ (e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell); a cell from an endocrine system or organ (e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte)); a cell from a nervous system or organ (e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph)); a cell from a respiratory system or organ (e.g., a pneumocyte (a type I pneumocyte and a type II pneumocyte), a clara cell, a goblet cell, an alveolar macrophage); a cell from circular system or organ (e.g., myocardiocyte and pericyte); a cell from digestive system or organ (e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell)); a cell from integumentary system or organ (e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell)), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A cell further includes a mammalian zygote or a stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A stem cell may also include a cancer stem cell. A mammalian cell can be a rodent cell, e.g., a mouse, rat, hamster cell. A mammalian cell can be a lagomorpha cell, e.g., a rabbit cell. A mammalian cell can also be a primate cell, e.g., a human cell.

The term “construct” or “nucleic acid construct” as used herein refers to a nucleic acid in which a polynucleotide sequence of interest is inserted into a vector. The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, and artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus (AAV), herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

The term “double-stranded” as used herein refers to one or two nucleic acid strands that have hybridized along at least a portion of their lengths. In certain embodiments, “double-stranded” does not mean that a nucleic acid must be entirely double-stranded. Instead, a double-stranded nucleic acid can have one or more single-stranded segment and one or more double-stranded segment. For example, a double-strand nucleic acid can be a double-strand DNA, a double-strand RNA, or a double-strand DNA/RNA compound. The form of the nucleic acid can be determined using common methods in the art, such as molecular band stained with SYBR green and distinguished by electrophoresis.

The term “deliver” or “delivered” or “delivering” in the context of inserting a nucleic acid sequence into a cell, means “transfection”, or “transformation”, or “transduction” and includes reference to the incorporation of a nucleic acid sequence into a eukaryotic or prokaryotic cell wherein the nucleic acid sequence may be present in the cell transiently or may be incorporated into the genome of the cell (e.g., chromosome, plasmid, plastid, or mitochondrial DNA), converted into an autonomous replicon. The construct of the present disclosure may be delivered into a cell using any method known in the art. Various techniques for transfecting animal cells may be employed, including, for example: microinjection, retrovirus mediated gene transfer, electroporation, transfection, or the like (see, e.g., Keown et al., Methods in Enzymology 1990, 185:527-537). In one embodiment, the construct is delivered to the cell via a virus.

The term “exon” refers to a nucleotide sequence within a gene that encodes a part of the final mature RNA produced by that gene after introns have been removed by RNA splicing. As used herein, an exon refers to both the DNA sequence within a gene and the corresponding sequence in RNA transcripts.

The term “genome editing enzyme” refers to an enzyme capable of altering or modifying the genetic sequence in a cell. Genome editing enzymes include, without limitation, site-specific nucleases (e.g., Cas9, ZFN, TALEN and meganuclease) and site-specific recombinases (e.g., Cre, FLP, lamda integrase, phiC31 integrase, Bxb1 integrase, gamma-delta resolvase, Tn3 resolvase and Gin invertase).

The term “intron” refers to a nucleotide sequence within a gene that is removed by RNA splicing during maturation of the final RNA product. The term “intron” refers to both the DNA sequence within a gene and the corresponding sequence in RNA transcripts.

The term “modification” or “genetic modification” refers to a disruption at the genomic level that may result in a decrease or increase in the expression or activity of a gene expressed by a cell. Exemplary modifications can include insertion, deletions, replacement, frame shift mutations, point mutations, exon removal, removal of one or more DNAse 1-hypersensitive sites (DHS) (e.g., 2, 3, 4 or more DHS regions), etc.

“Desired modification” in the context of gene-editing refers to the genetic modification of interest, which is pursued by the manipulator. The desired modification of the present disclosure can be a modification in the genomic region that is capable of recovering, enhancing, or changing the normal function or a selected function of a gene, or increasing or decreasing the expression of a gene. “Undesired modification” is opposite to “desired modification”, which is unwanted modification resulted from random modification that is different from those are desired. In certain embodiments of the present disclosure, one or more desired modification and/or one or more undesired modification of a genomic region can be generated by CRISPR-associated system.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, a “nuclease” is an enzyme capable of cleaving the phosphodiester bonds between the nucleotide subunits of nucleic acids. A “site-specific nuclease” refers to a nuclease whose functioning depends on a specific nucleotide sequence. Typically, a site-specific nuclease recognizes and binds to a specific nucleotide sequence and cuts a phosphodiester bond within the nucleotide sequence. In certain embodiments, the double-strand break is generated by site-specific cleavage using a site-specific nuclease. Examples of site-specific nucleases include, without limitation, zinc finger nucleases (ZFNs), transcriptional activator-like effector nucleases (TALENs), meganuclease and CRISPR (clustered regularly interspaced short palindromic repeats)-associated (Cas) nucleases.

A site-specific nuclease typically contains a DNA-binding domain and a DNA-cleavage domain. For example, a ZFN contains a DNA binding domain that typically contains between three and six individual zinc finger repeats and a nuclease domain that consists of the FokI restriction enzyme that is responsible for the cleavage of DNA. The DNA binding domain of ZFN can recognize between 9 and 18 base pairs. In the example of a TALEN, which contains a TALE domain and a DNA cleavage domain, the TALE domain contains a repeated highly conserved 33-34 amino acid sequence with the exception of the 12^{th} and 13^{th} amino acids, whose variation shows a strong correlation with specific nucleotide recognition. For another example, Cas9, a typical Cas nuclease, is composed of an N-terminal recognition domain and two endonuclease domains (RuvC domain and HNH domain) at the C-terminus.

The term “operably linked” refers to an arrangement of elements wherein the components so described are configured so as to perform their usual function. When used with respect to polynucleotides, the term refers to a juxtaposition, with or without a spacer or linker, of two or more polynucleotide sequences of interest in such a way that they are in a relationship permitting them to function in an intended manner. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide. The regulatory sequence need not be contiguous with the coding sequence, so long as they function to direct the expression thereof. For example, intervening untranslated yet transcribed sequences can be present between the regulatory sequence and the coding sequence and the regulatory sequence can still be considered “operably linked” to the coding sequence. For another example, the regulatory sequence may be contained within the coding sequence, e.g., within an intron, and the regulatory sequence can still be considered “operably linked” to the coding sequence.

As used herein, a “promoter” and “promoter-enhancer” sequence is an array of nucleic acid control sequences to which RNA polymerase binds and initiates transcription. A promoter includes necessary nucleic acid sequences near the start site of transcription, such as, in the case of a polymerase II type promoter, a TATA element. A promoter-enhancer also optionally includes distal enhancer or repressor elements which can be located as much as several thousand base pairs from the start site of transcription. The promoter determines the polarity of the transcript by specifying which DNA strand will be transcribed. Eukaryotic promoters are complex arrangements of sequences that are utilized by RNA polymerase II. General transcription factors (GTFS) first bind specific sequences near the start and then recruit the binding of RNA polymerase II. In addition to these minimal promoter elements, small sequence elements are recognized specifically by modular DNA-binding/trans-activating proteins (e.g., AP-1, SP-1) that regulate the activity of a given promoter. Viral promoters serve the same function as bacterial or eukaryotic promoters and either provide a specific RNA polymerase in trans (bacteriophage T7) or recruit cellular factors and RNA polymerase (SV40, RSV, CMV). Promoters may be, furthermore, either constitutive or regulatable. Inducible elements are DNA sequence elements which act in conjunction with promoters and may bind either repressors or inducers. In such cases, transcription is virtually “shut off” until the promoter is derepressed or induced, at which point transcription is “turned-on.” Examples of eukaryotic promoters include, but are not limited to, the following: the promoter of the mouse metallothionein I gene sequence (Hamer et al., J. Mol. Appl. Gen. (1982) 1:273-288); the TK promoter of Herpes virus (McKnight, Cell (1982) 31:355-365); the SV40 early promoter (Benoist et al., Nature (1981) 290:304-310); the yeast gall gene sequence promoter (Johnston et al., Proc. Natl. Acad. Sci. (1982) 79:6971-6975); Silver et al., Proc. Natl. Acad. Sci. (1984) 81:5951-59SS), the CMV promoter, the EF-1 promoter, Ecdysone-responsive promoter(s), tetracycline responsive promoter, and the like.

In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

As used herein, the term “recombinase” or “site-specific recombinase” refers to a family of highly specialized enzymes that promote DNA rearrangement between specific target sites (Greindley et al., 2006; Esposito, D., and Scocca, J. J., Nucleic Acids Research 25, 3605-3614 (1997); Nunes-Duby, S. E., et al, Nucleic Acids Research 26, 391-406 (1998); Stark, W. M., et al, Trends in Genetics 8, 432-439 (1992)). Virtually all site-specific recombinases can be categorized within one of two structurally and mechanistically distinct groups: the tyrosine (e.g., Cre, Flp, and the lambda integrase) or serine (e.g., phiC31 integrase, Bxb1 integrase, gamma-delta resolvase, Tn3 resolvase and Gin invertase) recombinases. Both recombinase families recognize target sites composed of two inversely repeated binding elements that flank a spacer sequence where DNA breakage and religation occur. The recombination process requires concomitant binding of two recombinase monomers to each target site: two DNA-bound dimers (a tetramer) then join to form a synaptic complex, leading to crossover and strand exchange.

As used herein, the term “riboswitch” refers to a regulatory segment of a messenger RNA molecule that binds a small molecule, resulting in a change in production of the proteins encoded by the mRNA. Riboswitches include, without limitation, Cobalamin riboswitch, cyclin AMP-GMP riboswitches, cyclic di-AMP riboswitches, cyclic di-GMP riboswitches, fluoride riboswitches, FMN riboswitches, glmS riboswitches, glutamine riboswitches, glycine riboswitches, lysine riboswitches, manganese riboswitches, NiCo riboswitches, PreQ1 riboswitches, purine riboswitches, SAH riboswitches, SAM riboswitches, SAM-SAH riboswitches, tetrahydrofolate riboswitches, TPP riboswitches, ZMP/ZTP riboswitches. In certain embodiment, the small molecule is a metabolite, such as a riboswitch metabolite, e.g., adenosylcobalamin, aquocobalamin, cAMP, cGMP, c-di-AMP, c-di-GMP, fluoride, falvin mononucleotide, glutamine, glycine, lysine, nickel, cobalt, pre-queuosine, purine, S-adenosyl methionine, tetrahydrofolate, thiamin pyrophosphate, guanine, adenine, 2′-deoxyguanosine, 7-aminomethyl-7-deazaguanine, ZMP and ZTP.

The term “subject” or “individual” or “animal” or “patient” as used herein refers to human or non-human animal, including a mammal or a primate, in need of diagnosis, prognosis, amelioration, prevention and/or treatment of a disease or disorder such as viral infection or tumor. Mammalian subjects include humans, domestic animals, farm animals, and zoo, sports, or pet animals such as dogs, cats, guinea pigs, rabbits, rats, mice, horses, swine, cows, bears, and so on.

In the context of formation of a CRISPR complex, “target” refers to a guide sequence (that is, gRNA) designed to have complementarity to a genomic region (that is, a target sequence), where hybridization between the genomic region and a guide RNA promotes the formation of a CRISPR complex. The terms “complementarity” or “complementary” are used in reference to polynucleotides (i.e., a sequence of nucleotides) related by the base-pairing rules. Complementarity may be “partial,” in which only some of the nucleic acids' bases are matched according to the base pairing rules (e.g., 5, 6, 7, 8, 9, 10 out of 10 being 50%, 60%, 70%, 80%, 90%, and 100% complementary), or there may be “complete” or “total” complementarity between the nucleic acids. The degree of complementarity between nucleic acid strands has significant effects on the efficiency and strength of their hybridization to one another.

“Transcript” or “RNA transcript” refers to an RNA molecule formed by the gene transcription for protein expression. RNA polymerase transcribes primary transcript mRNA (known as pre-mRNA), which is processed into mature mRNA. Therefore, RNA transcripts as used herein include both primary transcript mRNA and processed, mature mRNA. One or more transcripts variants may be formed from the same DNA segment via differential splicing. In such a process, particular exons of a gene may be included within or excluded from the messenger mRNA (mRNA), resulting in translated proteins containing different amino acids and/or possessing different biological functions.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

### II. GENOME EDITING ENZYMES

The present disclosure in one aspect relates to a controllable system for genome editing. In certain embodiments, the system is capable of switching the expression of a genome editing enzyme upon the presence or absence of an effector molecule.

In certain embodiments, genome editing enzymes include, without limitation, site-specific nucleases (e.g., Cas9, ZFN, TALEN and meganuclease) and site-specific recombinases (e.g., Cre, FLP, lamda integrase, phiC31 integrase, Bxb1 integrase, gamma-delta resolvase, Tn3 resolvase and Gin invertase).

CRISPR (Clustered Regularly Interspaced Short Palindromic Repeats)/Cas system was originally found as transcripts and other elements in the prokaryotic cells involved in the expression of or directing the activity of CRISPR-associated (“Cas”) genes, including sequences encoding a Cas nuclease that cleaves the nucleic acid sequence and generates double strand break (DSB), a guide sequence, a trans-activating CRISPR (tracr) sequence, a tracr-mate sequence, or other sequences and transcripts from a CRISPR locus. In eukaryotic cells, the CRISPR/Cas system comprises a CRISPR-associated nuclease and a small guide RNA. The target DNA sequence (the protospacer) contains a “protospacer-adjacent motif” (PAM), a short DNA sequence recognized by the particular Cas protein being used. In certain embodiments, the CRISPR system comprises CRISPR/Cas system of type I, type II, and type III, which comprises protein Cas3, Cas9 and Cas10, respectively.

The RNA-guided endonuclease Cas9 is a component of the type II CRISPR system widely utilized generate gene-specific knockouts in a variety of model systems. In one embodiment of the present disclosure, the CRISPR/Cas nuclease is a “sequence-specific nuclease”. Introduction of ectopic expression of Cas9 and a single guide RNA (gRNA) is sufficient to lead to the formation of double-strand breaks (DSBs) at a specific genomic region of interest, which leads to an indel via NHEJ pathway. Indels often result in frameshift mutations, except when the number of inserted/deleted nucleotides is a multiple of 3.

Along with Cas endonuclease, CRISPR experiments require the introduction of a guide RNA containing an approximately 15 to 30 base sequence specific to a target nucleic acid (e.g., DNA). A gRNA designed to target a genomic region of interest, for example, a particular exon encoding a functional domain of a protein, will generate a mutation in each gene that encodes the protein. The resulted modified genomic region may comprise one or more variants, each of which is different in the mutation. For example, the mutation will result in a modified genomic region with a desired modification, and/or a modified genomic region with an undesired modification. This approach has been widely utilized to generate gene-specific knockouts in a variety of model systems. In certain embodiments, a gRNA has a length of 15, 16, 17, 18, 19, 20, 21, 22, 23, 24, 25, 26, 27, 28, 29 or 30 nucleotides. gRNA can be delivered into an eukaryotic cell or a prokaryotic cell as RNA or by transfection with a vector (e.g., plasmid) having a gRNA-coding sequence operably linked to a promoter.

In certain embodiments, the Cas nuclease and the gRNA are derived from the same species. In certain embodiments, the Cas nuclease is derived from, for example, Staphylococcus aureus, Staphylococcus epidermidis, Staphylococcus sciuri, Pseudomonas aeruginosa, Enterococcus faecium, Enterococcus faecalis, Escherichia coli, Klebsiella pneumoniae, Streptococcus pneumoniae, Streptococcus pyrogenes, Lactobacillus bulgaricus, Streptococcus thermophilus, Vibrio cholera, Achromobacter xylosoxidans, Burkholderia cepacia, Citrobacter diversus, Citrobacter freundii, Micrococcus leuteus, Proteus mirabilis, Proteus vulgaris, Staphylococcus lugdunegis, Salmonella typhi, Streptococcus Group A, Streptococcus Group B, S. marcescens, Enterobacter cloacae, Bacillus anthracis, Bordetella pertussis, Clostridium sp., Clostridium botulinum, Clostridium tetani, Corynebacterium diphtheria, Moraxalla (Brauhamella) catarrhalis, Shigella spp., Haemophilus influenza, Stenotrophomonas maltophili, Pseudomonas perolens, Pseuomonas fragi, Bacteroides fragilis, Fusobacterium sp., Veillonella sp., Yersinia pestis, and Yersinia pseudotuberculosis.

A gRNA can be designed using any known software in the art, such as Target Finder, E-CRISPR, CasFinder, and CRISPR Optimal Target Finder.

In certain embodiments, the composition described herein comprises a nucleic acid encoding the Cas nuclease or the gRNA, wherein the nucleic acid is contained in a vector. In some embodiments, the composition comprises Cas nuclease protein and a DNA encoding the gRNA. In some embodiments, the composition comprises a first nucleic acid encoding the Cas nuclease and a second nucleic acid encoding the gRNA, whereas the first and the second nucleic acids are contained in one vector. In some embodiment, the first and the second nucleic acids are contained in two separate vectors. In some embodiments, at least one vector is a viral vector. In certain embodiments, the vector is AAV vector.

A zinc finger nuclease (ZFN) is an artificial restriction enzyme generated by fusing a zinc finger DNA-binding domain to a DNA-cleavage domain. Zinc finger domain can be engineered to target specific desired DNA sequences, which directs the zinc finger nucleases to cleave the target DNA sequences. Typically, a zinc finger DNA-binding domain contains three to six individual zinc finger repeats and can recognize between 9 and 18 base pairs. Each zinc finger repeat typically includes approximately 30 amino acids and comprises a ββα-fold stabilized by a zinc ion. Adjacent zinc finger repeats arranged in tandem are joined together by linker sequences. Various strategies have been developed to engineer zinc finger domains to bind desired sequences, including both “modular assembly” and selection strategies that employ either phage display or cellular selection systems (Pabo C O et al., “Design and Selection of Novel Cys2His2 Zinc Finger Proteins” Annu. Rev. Biochem. (2001) 70:313-40). The most straightforward method to generate new zinc-finger DNA-binding domains is to combine smaller zinc-finger repeats of known specificity. The most common modular assembly process involves combining three separate zinc finger repeats that can each recognize a 3 base pair DNA sequence to generate a 3-finger array that can recognize a 9 base pair target site. Other procedures can utilize either 1-finger or 2-finger modules to generate zinc-finger arrays with six or more individual zinc finger repeats. Alternatively, selection methods have been used to generate zinc-finger DNA-binding domains capable of targeting desired sequences. Initial selection efforts utilized phage display to select proteins that bound a given DNA target from a large pool of partially randomized zinc-finger domains. More recent efforts have utilized yeast one-hybrid systems, bacterial one-hybrid and two-hybrid systems, and mammalian cells. A promising new method to select novel zinc-finger arrays utilizes a bacterial two-hybrid system that combines pre-selected pools of individual zinc finger repeats that were each selected to bind a given triplet and then utilizes a second round of selection to obtain 3-finger repeats capable of binding a desired 9-bp sequence (Maeder M L, et al., “Rapid ‘open-source’ engineering of customized zinc-finger nucleases for highly efficient gene modification”. Mol. Cell (2008) 31(2): 294-301). The non-specific cleavage domain from the type II restriction endonuclease FokI is typically used as the cleavage domain in ZFNs. This cleavage domain must dimerize in order to cleave DNA and thus a pair of ZFNs are required to target non-palindromic DNA sites. Standard ZFNs fuse the cleavage domain to the C-terminus of each zinc finger domain. In order to allow the two cleavage domains to dimerize and cleave DNA, the two individual ZFNs must bind opposite strands of DNA with their C-termini a certain distance apart. The most commonly used linker sequences between the zinc finger domain and the cleavage domain requires the 5′ edge of each binding site to be separated by 5 to 7 bp.

A transcription activator-like effector nuclease (TALEN) is an artificial restriction enzyme made by fusing a transcription activator-like effector (TALE) DNA-binding domain to a DNA cleavage domain (e.g., a nuclease domain), which can be engineered to cut specific sequences. TALEs are proteins that are secreted by Xanthomonas bacteria via their type III secretion system when they infect plants. TALE DNA-binding domain contains a repeated highly conserved 33-34 amino acid sequence with divergent 12th and 13th amino acids, which are highly variable and show a strong correlation with specific nucleotide recognition. The relationship between amino acid sequence and DNA recognition allows for the engineering of specific DNA-binding domains by selecting a combination of repeat segments containing the appropriate variable amino acids. The non-specific DNA cleavage domain from the end of the FokI endonuclease can be used to construct TALEN. The FokI domain functions as a dimer, requiring two constructs with unique DNA binding domains for sites in the target genome with proper orientation and spacing. See Boch, Jens “TALEs of genome targeting” Nature Biotechnology (2011) 29: 135-6; Boch, Jens et al., “Breaking the Code of DNA Binding Specificity of TAL-Type III Effectors” Science (2009) 326: 1509-12; Moscou M J and Bogdanove A J “A Simple Cipher Governs DNA Recognition by TAL Effectors” Science (2009) 326 (5959): 1501; Juillerat A et al., “Optimized tuning of TALEN specificity using non-conventional RVDs” Scientific Reports (2015) 5: 8150; Christian et al., “Targeting DNA Double-Strand Breaks with TAL Effector Nucleases” Genetics (2010) 186 (2): 757-61; Li et al., “TAL nucleases (TALNs): hybrid proteins composed of TAL effectors and Fold DNA-cleavage domain” Nucleic Acids Research (2010) 39: 1-14.

Site-specific recombinases refer to a family of enzymes that mediate the site-specific recombination between specific DNA sequences recognized by the enzymes. Examples of site-specific recombinase include, without limitation, Cre recombinase, Flp recombinase, the lambda integrase, gamma-delta resolvase, Tn3 resolvase, Sin resolvase, Gin invertase, Hin invertase, Tn5044 resolvase, Tn3 transposase, sleeping beauty transposase, IS607 transposase, Bxb1 integrase, wBeta integrase, BL3 integrase, phiR4 integrase, A118 integrase, TG1 integrase, MR11 integrase, phi370 integrase, SPBc integrase, SV1 integrase, TP901-1 integrase, phiRV integrase, FC1 integrase, K38 integrase, phiBT1 integrase and phiC31 integrase.

### III. REGULATORY CASSETTE

The present disclosure in one aspect provides a regulatory expression construct which encodes an RNA that comprises a regulatory cassette controlling the expression of a sequence, i.e., the main coding region, operably linked to the regulatory cassette via binding to an effector molecule.

The regulatory cassette described herein is an expression control element that is part of the RNA molecule to be expressed and that changes state when bound by an effector molecule. In some embodiment, the regulatory cassette locates in the 5′-untranslated region of the main coding region. In some embodiment, the regulatory cassette locates in the 3′-untranslated region of the main coding region. In some embodiment, the regulatory cassette is inserted and locates within the main coding region.

Typically, the regulatory cassette comprises two separate domains: an aptamer domain that selectively binds the effector molecule and an expression platform domain that influences genetic control. The dynamic interplay between the two domains results in the control of gene expression depending on the presence of the effector molecule. Disclosed herein are isolated and recombinant regulatory cassette, recombinant constructs containing such regulatory cassette, heterologous sequences operably linked to such regulatory cassette, and cells and transgenic organisms harboring such regulatory cassette. The heterologous sequences can be, for example, sequences encoding proteins or peptides of interest, including genomic editing enzymes.

The disclosed regulatory cassette, including the derivatives and recombinant forms thereof, generally can be from any source, including naturally occurring regulatory cassette and those designed de novo. Any such regulatory cassettes can be used in or with the disclosed methods. A naturally occurring regulatory cassette is a regulatory cassette having the sequence of a regulatory cassette, e.g., a riboswitch as found in nature. Such a naturally occurring regulatory cassette can be an isolated or recombinant form of the naturally occurring regulatory cassette as it occurs in nature. That is, the regulatory cassette has the same primary structure but has been isolated or engineered in a new genetic or nucleic acid context. Chimeric regulatory cassette can be made up of, for example, part of a regulatory cassette of any or of a particular class or type of regulatory cassette and part of a different regulatory cassette of the same or of any different class or type of regulatory cassette; part of a regulatory cassette of any or of a particular class or type of regulatory cassette and any non-regulatory cassette sequence or component. Recombinant regulatory cassettes are those that have been isolated or engineered in a new genetic or nucleic acid context.

1. Aptamer Domain

Aptamers are nucleic acid segments and structures that can bind selectively to particular compounds and classes of compounds. The regulatory cassettes described herein have aptamer domains that, upon binding of an effector molecule result in a change the state or structure of the regulatory cassette. In certain embodiments, the state or structure of the expression platform domain linked to the aptamer domain changes when the effector molecule binds to the aptamer domain. Aptamer domains of the regulatory cassettes described herein can be derived from any source, including, for example, naturally-occurring aptamer domains, artificial aptamers, engineered, selected, evolved or derived aptamers or aptamer domains. Aptamers in the regulatory cassettes described herein generally have at least one portion that can interact, such as by forming a stem structure, with a portion of the linked expression platform domain. This stem structure will either form or be disrupted upon binding of the effector molecule.

Suitable methods for generating the aptamer domains used in the present application has been described in the art. For example, one method for generating an aptamer is with the process entitled “Systematic Evolution of Ligands by Exponential Enrichment” (“SELEX™”) described in, e.g., U.S. Pat. Nos. 5,475,096, and 5,270,163. The SELEX™ process is a method for the in vitro evolution of nucleic acid molecules with highly specific binding to target molecules. Each SELEX™-identified nucleic acid ligand, i.e., each aptamer, is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (i.e., form specific binding pairs) with virtually any chemical compound, whether monomeric or polymeric. Molecules of any size or composition can serve as targets.

In general, the SELEX™ methods start with a large library or pool of single stranded oligonucleotides comprising randomized sequences. The oligonucleotides can be modified or unmodified DNA, RNA, or DNA/RNA hybrids. In some examples, the pool comprises 100% random or partially random oligonucleotides. In other examples, the pool comprises random or partially random oligonucleotides containing at least one fixed and/or conserved sequence incorporated within randomized sequence which can be used as, e.g., hybridization sites for PCR primers, promoter sequences for RNA polymerases, restriction sites, or homopolymeric sequences, to facilitate cloning and/or sequencing of an oligonucleotide of interest.

Typically, the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis before or during the selection/amplification iterations.

Within the starting pool containing a large number of possible sequences and structures, there is a wide range of binding affinities for a given target. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

Some examples of the aptamer domain have been described previous (see U.S. Pat. No. 7,794,931 to Breaker et al., the disclosure of which is incorporated herein by reference). In particular, Vogel M et al. have disclosed a synthetic riboswitch that efficiently controls alternative splicing of a cassette exon in response to the small molecule ligand tetracycline. In the presence of tetracycline, the cassette exon is skipped, whereas it is included in the ligand's absence (Nucleic Acid Research (2018) 46:e48).

In certain embodiments, the aptamer domain has a sequence of at least 90% (e.g. 90%, 95%, 98%, 99%) identity to SEQ ID NO: 16, 18 or 20 in the DNA form or SEQ ID NO: 17, 19 or 21 in the RNA form.

2. Expression Platform Domain

Expression platform domains are a part of the regulatory cassettes described herein that affect expression of the RNA molecule that contains the regulatory cassettes. Generally, expression platform domains have at least one portion that can interact, such as by forming a stem structure, with a portion of the linked aptamer domain. This stem structure will either form or be disrupted upon binding of the effector molecule. The stem structure generally either is, or prevents formation of, an expression regulatory structure. An expression regulatory structure is a structure that allows, prevents, enhances or inhibits expression of an RNA molecule containing the structure. Examples of the expression platform domain include Shine-Dalgarno sequences, initiation codons, transcription terminators, introns, exons, and stability and processing signals.

In certain embodiments, the expression platform domain comprises a conditional exon flanked by an upstream intron and a downstream intron. In one embodiment, the conditional exon has a sequence of at least 90% (e.g. 90%, 95%, 98%, 99%) identity to SEQ ID NO: 22 in the DNA form or SEQ ID NO: 23 in the RNA form. In one embodiment, the upstream intron has a sequence of at least 90% (e.g. 90%, 95%, 98%, 99%) identity to SEQ ID NO: 24 in the DNA form or SEQ ID NO: 25 in the RNA form. In one embodiment, the downstream intron has a sequence of at least 90% (e.g. 90%, 95%, 98%, 99%) identity to SEQ ID NO: 26 in the DNA form or SEQ ID NO: 27 in the RNA form.

3. Effector Molecules

Effector molecules as used herein are molecules and compounds that can activate a regulatory cassette. This includes the natural or normal effector molecule for the naturally-occurring regulatory cassette, e.g. a riboswitch, and other compounds that can activate the regulatory cassette. In the case of some synthetic regulatory cassette, the effector molecule can be those for which the aptamer domain is designed or with which the aptamer domain was selected (as in, for example, in vitro selection or in vitro evolution techniques).

In certain embodiments, the effector molecule is tetracycline. In certain embodiments, the effector molecule is a metabolite, e.g., adenosylcobalamin, aquocobalamin, cAMP, cGMP, c-di-AMP, c-di-GMP, fluoride, falvin mononucleotide, glutamine, glycine, lysine, nickel, cobalt, pre-queuosine, purine, S-adenosyl methionine, tetrahydrofolate, thiamin pyrophosphate, guanine, adenine, 2′-deoxyguanosine, 7-aminomethyl-7-deazaguanine, ZMP and ZTP.

4. Embodiments of Regulatory Cassettes

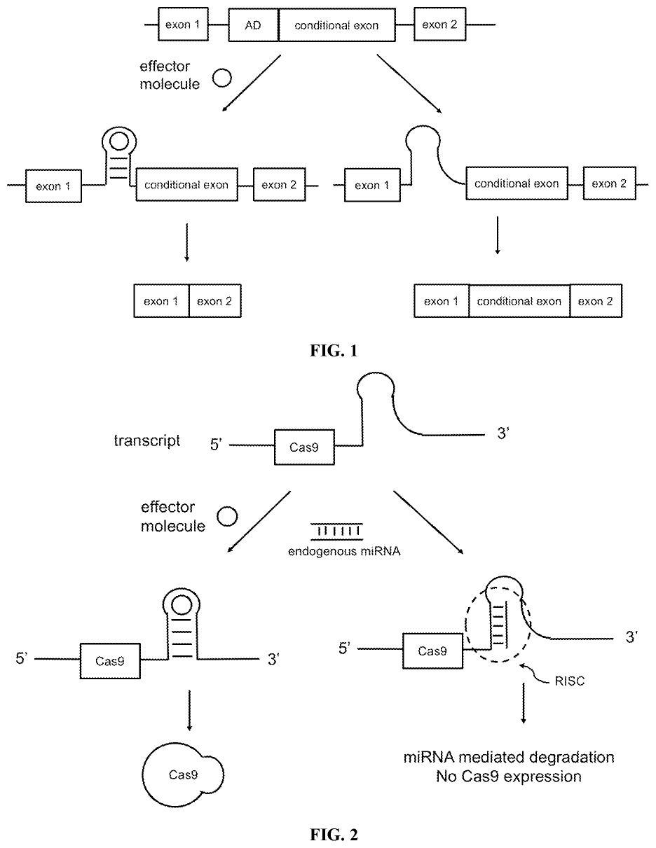

FIG. 1 illustrates an exemplary embodiment of the regulatory cassette of the present invention in controlling the expression of a genome editing enzyme via alternative splicing of a conditional exon. Referring to FIG. 1, a regulatable gene expression construct comprises a polynucleotide sequence encoding a genome editing enzyme. The polynucleotide sequence includes exon 1 of the genome editing enzyme, exon 2 of the genome editing enzyme and a conditional exon interspersed between exon 1 and exon 2. The conditional exon does not encode part of the genome editing enzyme but includes a stop codon. The conditional exon is preceded by a regulatory sequence encoding an aptamer domain (AD) capable of changing its structure upon binding to an effector molecule. When the DNA construct is delivered into a cell, the DNA construct is transcribed into an RNA transcript. In the presence of the effector molecule, the aptamer domain binds to the effector molecule and forms a structure that block the splicing acceptor of the conditional exon. As a result, the RNA transcript is spliced into a mature mRNA that includes only exon 1 and exon 2 and is translated to functional genome editing enzyme. In the absence of the effector molecule, the aptamer domain forms a structure that does not block the splicing acceptor site of the conditional exon. As a result, the RNA transcript is spliced into a mature mRNA that includes exon1, conditional exon and exon 2. The resulted mRNA is not translated to a functional genome editing enzyme.

FIG. 2 illustrates an exemplary embodiment of the regulatory cassette of the present invention in controlling the expression of a genome editing enzyme via regulating the stability of the RNA transcript. Referring to FIG. 2, a regulatable gene expression construct encodes an RNA that includes a polynucleotide sequence encoding a genome editing enzyme (e.g., Cas9) and a regulatory cassette operably linked to the 3′ end of the polynucleotide sequence. The regulatory cassette includes an aptamer domain capable of changing structure upon binding to an effector molecule. The regulatory cassette further includes a region that can be recognized by an endogenous miRNA. When the nucleic acid construct is delivered into a cell, the nucleic acid construct is transcribed into an RNA transcript comprising a region encoding the genome editing enzyme followed by the regulatory cassette. In the presence of the effector molecule, the aptamer domain binds to the effector molecule, and the regulatory cassette forms a stem loop structure that is not recognized by the endogenous miRNA. As a result, the RNA transcript is translated to a functional genome editing enzyme. In the absence of the effector molecule, the aptamer domain does not form a stem loop, and the regulatory cassette is recognized by the endogenous miRNA, which leads to the degradation of the RNA transcript, e.g., through RISC pathway. As a result, the genome editing enzyme is not expressed.

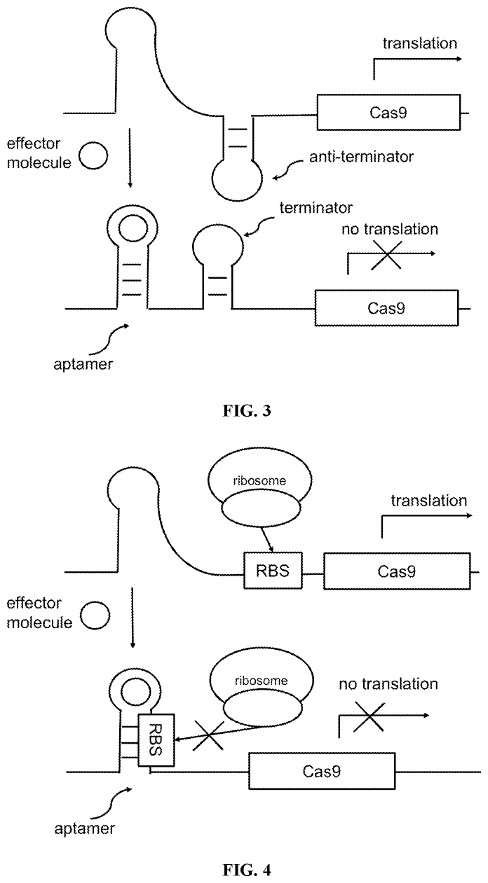

FIG. 3 illustrates an exemplary embodiment of the regulatory cassette of the present invention in controlling the expression of a genome editing enzyme via regulating the translation of the RNA transcript. Referring to FIG. 3, a regulatable gene expression construct encodes an RNA that includes a polynucleotide sequence encoding a genome editing enzyme (e.g., Cas9) and a regulatory cassette operably linked to the 5′ end of the polynucleotide sequence. The regulatory cassette includes an aptamer domain and a expression platform domain that forms an anti-terminator stem when the aptamer domain does not bind to an effector molecule and is capable of forming a terminator upon binding to the effector molecule. When the regulatable gene expression construct is delivered into a cell, the construct is transcribed into an RNA transcript comprising a region encoding the genome editing enzyme. In the absence of the effector molecule, the regulatory cassette forms an anti-terminator stem. As a result, the RNA transcript is translated to a functional genome editing enzyme. In the presence of the effector molecule, the aptamer domain binds to the effector molecule, and the regulatory cassette forms a terminator. As a result, the genome editing enzyme is not translated.

FIG. 4 illustrates another exemplary embodiment of the regulatory cassette of the present invention in controlling the expression of a genome editing enzyme via regulating the translation of the RNA transcript. Referring to FIG. 4, a regulatable gene expression construct encodes an RNA that includes a polynucleotide sequence encoding a genome editing enzyme (e.g., Cas9) and a regulatory cassette operably linked to the 5′ end of the polynucleotide sequence. The regulatory cassette includes an aptamer domain and is capable of forming a structure that sequesters the ribosome binding sequence (RBS) from being recognized by ribosome when the aptamer domain binds to an effector molecule. When the construct is delivered into a cell, the construct is transcribed into an RNA transcript comprising a region encoding the genome editing enzyme. In the absence of the effector molecule, the regulatory cassette forms a structure that allows the RBS to be recognized by ribosome. As a result, the RNA transcript is translated to a functional genome editing enzyme. In the presence of the effector molecule, the aptamer binds to the effector molecule and forms a structure that sequesters the RBS from being recognized by ribosome. As a result, the genome editing enzyme is not translated.

It is understood that the mechanisms described in the embodiments above can be used in combination. For example, the DNA construct can encode an RNA that comprise a polynucleotide sequence encoding a Cas9 as described in FIG. 1. The polynucleotide sequence includes exon 1 encoding the 5′ segment of Cas9 protein and exon 2 encoding the 3′ segment of Cas9 protein. Exon 1 and exon 2 are interspersed with a first regulatory cassette including a conditional exon. The conditional exon is preceded by a first aptamer domain capable of changing its structure upon binding to tetracycline. Exon 2 is followed by a second regulatory cassette including a second aptamer domain that is capable of forming a stem loop structure upon binding to tetracycline a region that can be recognized by an endogenous miRNA. When the DNA construct is delivered into a cell, the DNA construct is transcribed into an RNA transcript comprising exon 1, the first aptamer domain, the conditional exon, exon 2 and the second aptamer domain.

In the absence of tetracycline, the first aptamer domain forms a structure that does not block the splicing acceptor site of the conditional exon. As a result, the RNA transcript is spliced into a mature mRNA that includes exon1, conditional exon and exon 2. The resulted mRNA is not translated to a functional Cas9 protein. Meanwhile, the second aptamer domain does not form a stem loop and is recognized by the endogenous miRNA, which leads to the degradation of the RNA transcript through RISC pathway. As a result, Cas9 is not expressed.

In the presence of tetracycline, the first aptamer domain binds to tetracycline and forms a structure that blocks the splicing acceptor of the conditional exon. As a result, the RNA transcript is spliced into a mature mRNA that includes only the exon 1 and exon 2 and is translated to functional Cas9 protein. Meanwhile, the second aptamer domain binds to tetracycline and forms a stem loop structure that is not recognized by the endogenous miRNA. As a result, the RNA transcript is translated to a functional Cas9 protein.

### IV. COMPOSITIONS AND METHODS FOR CONTROLLABLE GENOME EDITING

1. Compositions

The disclosed regulatory cassette can be used in with any suitable expression system. Recombinant expression is usefully accomplished using a vector, such as a plasmid. The vector can include a promoter operably linked to regulatory cassette-encoding sequence and RNA to be expression (e.g., RNA encoding a protein). The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying the regulatable gene expression constructs can be produced. Such expression vectors include, for example, pET, pET3d, pCR2.1, pBAD, pUC, and yeast vectors. The vectors can be used, for example, in a variety of in vivo and in vitro situation.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Retroviral vectors, which are described in Verma (1985), include Murine Maloney Leukemia virus, MMLV, and retroviruses that express the desirable properties of MMLV as a vector. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

In certain embodiments, the regulatable gene expression construct also includes elements that enhances or facilitates the expression of the target gene. In certain embodiments, the regulatable gene expression construct includes a sequence encoding a nuclear localization signal (NLS) fused to the target gene that facilitates the expressed target protein to enter the nuclear. In certain embodiment, the NLS is a SV40 NLS or a nucleoplasmin NLS. In certain embodiments, the sequence encoding the NLS is SEQ ID NO: 36 or 38.

In certain embodiments, the regulatable gene expression construct also includes a sequence encoding a tag fused to the target protein to be expressed. In certain embodiments, the tag is an HA tag. In certain embodiments, the sequence encoding the tag is SEQ ID NO: 40.

In some embodiments, the regulatable gene expression construct also includes a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, mycophenolic acid, or hygromycin.

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

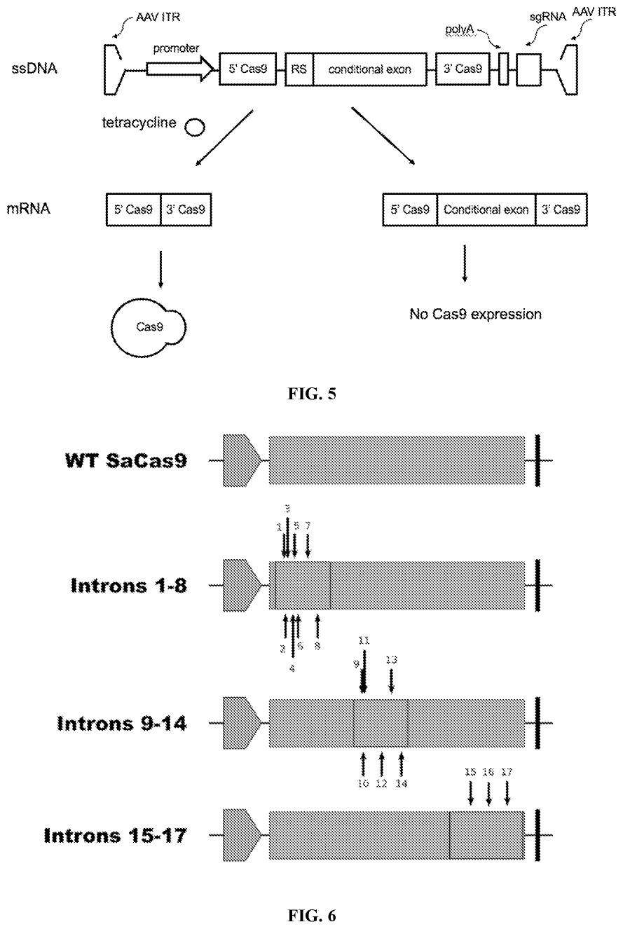

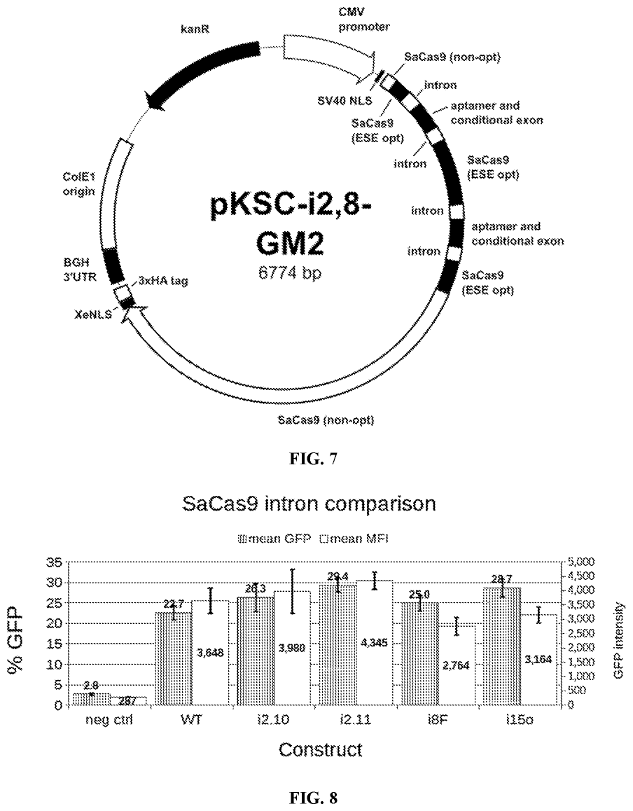

FIG. 5 illustrates a preferred embodiment in which the regulatable gene expression construct encodes a Cas9 protein and is included in an AAV vector. Referring to FIG. 5, the regulatable gene expression construct includes elements of an AAV vector, e.g., AAV inverted terminal repeats (ITR), a promoter and polyA region that control the expression of Cas9. The construct may also include a polynucleotide sequence encoding a guide RNA (sgRNA). The nucleic acid construct includes exon 1 encoding the 5′ segment of Cas9 protein and exon 2 encoding the 3′ segment of Cas9 protein. The construct also includes a sequence encoding a regulatory cassette including an aptamer domain followed by a conditional exon interspersed the first and the second region. The aptamer domain is capable of changing the structure of the regulatory cassette upon binding to tetracycline. When the regulatable gene expression construct is delivered into a cell, the construct is transcribed into an RNA transcript comprising the first region, the aptamer domain, the conditional exon and the second region. In the presence of tetracycline, the aptamer domain binds to tetracycline and forms a structure that blocks the splicing acceptor of the conditional exon. As a result, the RNA transcript is spliced into a mature mRNA that includes only the exon 1 and exon 2 and is translated to functional Cas9 protein. In the absence of tetracycline, the aptamer domain forms a structure that does not block the splicing acceptor site of the conditional exon. As a result, the RNA transcript is spliced into a mature mRNA that includes exon1, conditional exon and exon 2. The resulted mRNA is not translated to a functional Cas9 protein.

The regulatable gene expression construct described above as well as other materials can be packaged together in any suitable combination as a kit useful for performing, or aiding in the performance of, the disclosed method. It is useful if the kit components in a given kit are designed and adapted for use together in the disclosed method.

2. Methods

The present disclosure also provides uses the regulatable gene expression construct and compositions described herein. Disclosed are methods for regulating the expression of a target gene, e.g., a genome editing enzyme. Such methods can involve, for example, bringing into contact a regulatory cassette and an effector molecule that can activate, deactivate or block the regulatory cassette. Regulatory cassettes function to control gene expression through the binding or removal of an effector molecule. The expression of a target gene can also be controlled by, for example, removing effector molecules from the presence of the regulatory cassette. Thus, the disclosed method of regulating gene expression can involve, for example, removing an effector molecule from the presence or contact with the regulatory cassette. A regulatory cassette can be blocked by, for example, binding of an analog of the effector molecule that does not activate the regulatory cassette.

Also disclosed are methods of genome editing in a cell. In one embodiment, the method comprises delivering the regulatable gene expression construct that includes a sequence encoding a genome editing enzyme into the cell. In one embodiment, the method further comprises delivering the effector molecule to the cell. By switching the condition between the presence of absence of the effector molecule, the regulatory cassette is capable of turning on and off the expression of the genome editing enzyme, thus controlling the gene editing process mediated by the genome editing enzyme.

Also disclosed are methods of treating a subject having a disease. In one embodiment, the method comprises delivering the regulatable gene expression construct encoding a genome editing enzyme into at least one cell of the subject. In one embodiment, the method further comprises administering the effector molecule to the subject.

The diseases that can be treated by method disclosed herein include, without limitation, cancer, cystic fibrosis, heart disease, diabetes, hemophilia and AIDS.

### V. SEQUENCE SIMILARITIES

It is understood that as discussed herein the use of the terms homology and identity mean the same thing as similarity. Thus, for example, if the use of the word homology is used between two sequences (non-natural sequences, for example) it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed regulatory cassettes, aptamer domains, expression platform domains, genes and proteins herein, is through defining the variants and derivatives in terms of homology to specific known sequences. This identity of particular sequences disclosed herein is also discussed elsewhere herein. In general, variants of regulatory cassettes, aptamer domain, expression platform domains, introns, exons, genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to a stated sequence or a native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### VI. EXAMPLES

The following examples are included to demonstrate illustrative embodiments of the invention. It should be appreciated by those of skill in the art that the techniques disclosed in the examples which follow represent techniques discovered by the inventor to function well in the practice of the invention, and should only be considered to constitute illustrative modes for its practice. Those of skill in the art should, in light of the present disclosure, appreciate that many changes can be made in the specific embodiments which are disclosed and still obtain a like or similar result without departing from the spirit and scope of the invention.

### Example 1

This example illustrates the generation of a SaCas9 construct with addition of intron. While Cas9 gene is identified in bacteria, it has no natural introns and exons. To generate a Cas9 gene with an intron that can be properly transcribed and spliced, the inventors optimized three regions (SEQ ID NO: 10, 12 and 14) of Staphylococcus aureus Cas9 (SaCas9) gene (SEQ ID NO: 2) with enrichment of exonic splicing enhancer (ESE) and depletion of exonic splicing silencer (ESS). The inventors then generated a series of candidate SaCas9 genes, each having an intron inserted into one of the regions optimized with ESE enrichment and ESS depletion (FIG. 6). The candidate SaCas9 genes were cloned into a vector under CMV promoter.

The activity of candidate SaCas9 genes were then tested in an EGxxFP assay as described by Mashiko D et al. (see Sci Rep (2013) 3:3355). In short, the pCAG-EGxxFP plasmid containing 5′ and 3′ EGFP fragments that shares 482 bp under ubiquitous CAG promoter was prepared. An approximately 500 bp region containing the sgRNA target sequence was placed between EGFP fragments of pCAG-EGxxFP plasmid. The pCAG-EGxxFP plasmid was cotransfected with the candidate SaCas9 construct and sgRNA into HEK293T cells. When the candidate SaCas9 gene is properly transcribed and spliced, the target sequence in the EGxxFT gene was digested by sgRNA guided SaCas9 protein, the homologous dependent repair took place and reconstituted the EGFP expression.

As shown in FIG. 8, the results of the EGxxFP assay showed that positions 2, 8 and 15 are the best positions to insert an intron.

### Example 2

This example illustrates the insertion of an intron with a conditional exon regulated by an aptamer to a Cas9 gene.

After identified the positions in the SaCas9 gene to insert an intron, the inventors then tested three tetracycline aptamer domains M2 (SEQ ID NO: 16), M3 (SEQ ID NO: 18) and M4 (SEQ ID NO: 20) to control the splicing of a conditional exon. Candidate SaCas9 genes containing a tetracycline aptamer and conditional exon (SEQ ID NO: 22) flanked by two introns (SEQ ID NOs: 24 and 26) inserted in position 2 and 8 were prepared by inserted into vector. The candidate SaCas9 constructs were then tested in the EGxxFP assay as described in Example 1.

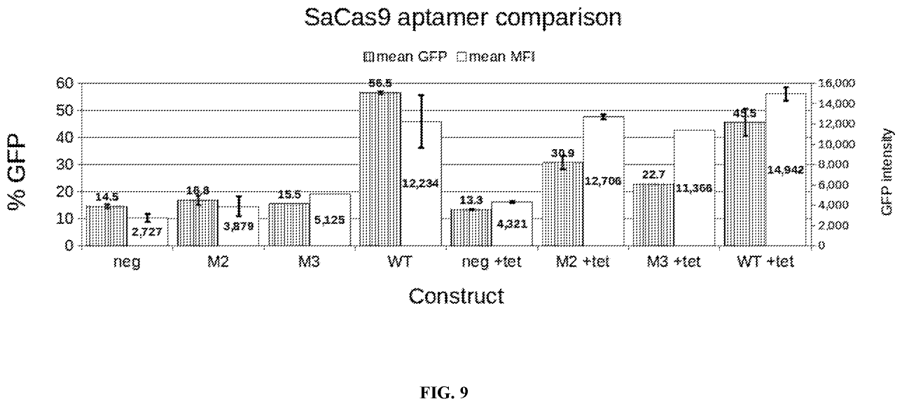

As shown in FIG. 9, the results of the EGxxFP assay showed that both M2 and M3 worked well in regulating the expression of SaCas9 while M2 performed the best.

### Example 3

This example illustrates the generation of a SaCas9 construct with dual aptamer in order to further repress the activity of SaCas9 in the absence of tetracycline.

To generate the candidate SaCas9 gene with two aptamer domains (SEQ ID NO: 34), the inventors inserted a tetracycline aptamer domain M2 and conditional exon into position 2 and a tetracycline aptamer domain M2 and conditional exon into position 8. The candidate SaCas9 gene with dual aptamer was then tested in the EGxxFP assay as described in Example 1.

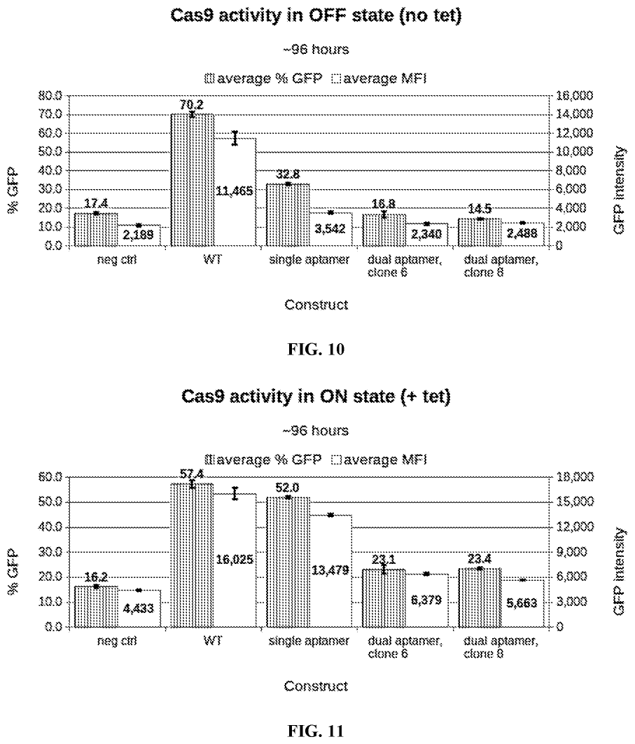

The results of the EGxxFP assay showed that the 2+8 dual aptamer gene has no activity above background in the absence of tetracycline (FIG. 10) and about 40% activity as compared to wildtype SaCas9 after 3 days in the presence of tetracycline (FIG. 11).

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1. A regulatable gene expression construct comprising a nucleic acid encoding an RNA, the RNA comprising
(1) a sequence encoding a genome editing enzyme, and
(2) a regulatory cassette operably linked to the sequence, the regulatory cassette comprising
(i) a conditional exon flanked by an upstream intron and a downstream intron, and
(ii) an aptamer domain operably linked to the conditional exon, wherein the aptamer domain is capable of binding to an effector molecule to trigger a structural change of the RNA, thereby regulating splicing of the conditional exon and expression of the genome editing enzyme.

2. The construct of claim 1, wherein the genome editing enzyme is expressed in the presence of the effector molecule.

3. The construct of claim 1, wherein the conditional exon is skipped during the splicing in the presence of the effector molecule.

4. The construct of claim 1, wherein the effector molecule is tetracycline.

5. The construct of claim 1, wherein the sequence is optimized to comprise an exonic splicing enhancer.

6. The construct of claim 1, wherein the genome editing enzyme is a site-specific nuclease or a site-specific recombinase, wherein the site-specific nuclease is selected from a group consisting of Cas9, Cas12, ZFN, TALEN and meganuclease and the site-specific recombinase is selected from a group consisting of Cre, FLP, lamda integrase, phiC31 integrase, Bxb1 integrase, gamma-delta resolvase, Tn3 resolvase and Gin invertase.

7-8. (canceled)

9. The construct of claim 1, wherein the genome editing enzyme has a sequence of at least 90% identity to SEQ ID NO: 1.

10. The construct of claim 1, wherein the sequence has at least 90% identity to SEQ ID NO: 5, 7 or 9, or the sequence comprises an exonic splicing enhancer (ESE) optimized region having at least 90% identity to SEQ ID NO: 11, 13 or 15.

11. (canceled)

12. The construct of claim 1, wherein the aptamer domain has a sequence of at least 90% identity to SEQ ID NO: 17, 19 or 21.

13. The construct of claim 1, wherein the conditional exon has a sequence of at least 90% identity to SEQ ID NO: 23.

14. The construct of claim 1, wherein the upstream intron has a sequence of at least 90% identity to SEQ ID NO: 25.

15. The construct of claim 1, wherein the downstream intron has a sequence of at least 90% identity to SEQ ID NO: 27.

16. The construct of claim 1, wherein the regulatory cassette comprises a sequence of at least 90% identity to SEQ ID NO: 29

17. The construct of claim 1, wherein the regulatory cassette is inserted between
(1) nucleotide position 97 and 98 of SEQ ID NO: 11; or
(2) nucleotide position 498 and 499 of SEQ ID NO: 11.

18. The construct of claim 1, comprising SEQ ID NO: 30, 32 or 34.

19. The construct of claim 1, which is contained in a vector wherein the vector is an AAV vector.

20. (canceled)

21. The construct of claim 1, wherein the gene editing enzyme is Cas9, and wherein the construct comprises a second polynucleotide sequence encoding a gRNA.

22. A method of genome editing in a cell, the method comprising delivering the construct of claim 1 into the cell, and further comprising delivering the effector molecule to the cell.

23. (canceled)

24. A modified cell made by delivering the construct of claim 1 into the cell.

25. A method of treating a subject having a disease, the method comprising delivering the construct of claim 1 into at least one cell of the subject, and further comprising, administering, the effector molecule to the subject.

26. (canceled)
